(19) United States
(12) Patent Application Publication
(10) Pub. No.: US 2018/0319838 A1
(43) Pub. Date: Nov. 8, 2018

(54) CONJUGATION METHODS FOR MODIFYING OR IMMOBILIZING PROTEINS

(71) Applicant: Biogen MA Inc., Cambridge, MA (US)

(72) Inventors: Carl CO, Somerville, MA (US); John E. COUGHLIN, Auburn, MA (US)

(73) Assignee: Biogen MA Inc., Cambridge, MA (US)

(21) Appl. No.: 15/773,739

(22) PCT Filed: Nov. 4, 2016
(86) PCT No.: PCT/US16/60651

§ 371 Date: May 4, 2018

Related U.S. Application Data
(60) Provisional application No. 62/251,025, filed on Nov. 4, 2015.

(51) Int. Cl. C07K 1/22; C12N 11/02; B01J 20/286; B01J 20/32; B01D 15/38; B01D 15/20

(52) U.S. Cl. CPC C07K 1/22; C12N 11/02; C12Y 205/01018; B01D 15/20; B01J 20/3217; B01D 15/3814; B01J 20/286

(57) ABSTRACT

The present disclosure relates, in some aspects, to protein-ligand localized conjugation technology with respect to immobilized functional proteins for affinity enrichment and/or modified proteins for therapeutic applications.

### RELATED APPLICATIONS

This application claims priority under 35 U.S.C. § 119(e) to U.S. provisional application No. 62/251,025, filed Nov. 4, 2015, the contents of which are incorporated herein in their entirety.

### FIELD OF THE INVENTION

The present disclosure relates, in some aspects, to the field of protein immobilization and protein modification.

### BACKGROUND OF INVENTION

The conjugation of proteins to solid supports and other molecules is a commonly used procedure in biochemistry laboratories. Such immobilized proteins can be used for affinity purification¹⁻³ or linked to molecules such as polyethylene glycol (PEG) to increase therapeutic efficacy.⁴ Protein attachment to solid supports may involve either non-covalent interactions or covalent chemistries. Molecular tags, such as poly-histidine residues or Glutathione-S-Transferase (GST) which are genetically attached to either the C- or N-terminus of the protein, can be used as molecular tags that can bind reversibly to Immobilized Metal Ion Chromatography resin (IMAC) and glutathione (GSH)-Sepharose respectively.⁵

### SUMMARY OF INVENTION

Aspects of the present disclosure provide methods and compositions for attaching a protein of interest to a substrate (e.g., a solid support, a polymer, or other molecule) without introducing unwanted modifications into the protein of interest. In some embodiments, methods are described for using a crosslinking agent to connect a protein of interest to a substrate without introducing unwanted crosslinks into the protein of interest.

In some aspects, a protein of interest is connected to a substrate via a covalently linked pair of binding partners (e.g., two molecules that bind to each other with high affinity and/or specificity), wherein one of the binding partners is attached to the protein of interest and the other binding partner is attached to the substrate.

In some embodiments, a protein of interest is connected to a substrate in a process involving two or more steps. In one step, a crosslinking agent is contacted to a first binding partner under first reaction conditions suitable for forming a first covalent bond between the crosslinking agent and the first binding partner, wherein the first binding partner is attached to a substrate. This produces a primed first binding partner that is attached to a substrate and that is covalently connected to a crosslinking agent capable of reacting with another molecule (e.g., a second binding partner) and forming a second covalent bond with that molecule. In a further step, a second binding partner is contacted to the first binding partner under second reaction conditions suitable for forming a second covalent bond between the crosslinking agent and the second binding partner, wherein the first and second binding partners bind to each other under the second reaction conditions, and wherein the second binding partner is attached to the protein of interest. In some embodiments, unbound crosslinking agent (e.g., crosslinking agent that did not form a covalent bond with the first binding partner) is removed from the reaction with the first binding partner before the second binding is added. This reduces the likelihood of random crosslinking of the protein of interest by unbound crosslinking agent remaining in the reaction. The primary crosslinking that should occur after adding the second binding partner is between the primed first binding partner and the second binding partner. It should be appreciated that the first and second binding partners can be chosen to have reactive groups (e.g., amino acid side chains) that are i) capable of reacting with a crosslinking agent, and ii) that are sufficiently near each other to allow crosslinking of the two binding partners after they bind to each other.

In some embodiments, the substrate connected to the first binding partner is a solid support. In some embodiments, the solid support comprises a resin. In some embodiments, the solid support comprises sepharose, agarose, silica, or polystyrene-divinyl-benzene. In some embodiments, the solid support is a sepharose bead.

In some embodiments, the substrate connected to the first binding partner is a polymer. In some embodiments, the polymer comprises polyethylene glycol (PEG).

In some embodiments, the crosslinking agent is a zero-length crosslinker. In some embodiments, the crosslinking agent covalently links a carboxylic acid to a primary amine. In some embodiments, the crosslinking agent is 1-ethyl-3-(dimethylaminopropyl) carbodiimide (EDC) or dicyclohexylcarbodiimide (DCC). In some embodiments, the crosslinking agent covalently links a primary amine to a primary amine. In some embodiments, the crosslinking agent is a N-hydroxysuccinimide (NHS)-ester crosslinker, or disuccinimidyl suberate (DSS).

In some embodiments, the substrate is covalently attached to the first binding partner. In some embodiments, the substrate is non-covalently attached to the first binding partner.

In some embodiments, the first binding partner is glutathione (GSH) and the second binding partner is glutathione S-transferase (GST), structural maintenance of chromosomes 1 (SMC1), or RalA Binding Protein 1 (RALBP1). In some embodiments, the second binding partner is a polypeptide.

In some embodiments, a protein of interest and the second binding partner are provided as a fusion protein (for example, they were expressed as a fusion protein from a recombinant gene encoding both the protein of interest and the second binding partner as a single polypeptide). In some embodiments, the second binding partner is attached to the N-terminus of the protein of interest. In some embodiments, the second binding partner is attached to the C-terminus of the protein of interest.

Accordingly, in some embodiments, a protein of interest is covalently attached to a solid support, a polymer, or other molecule via the covalent crosslinking of two non-covalent binding partners. In some embodiments, a solid support, a polymer, or other molecule is covalently linked to a first binding partner. In some embodiments, a protein of interest is covalently linked (e.g., as a protein fusion) to a second binding partner. In some embodiments, the first and second binding partners are covalently crosslinked to each other, thereby covalently connecting the protein of interest to the solid support, polymer, or other molecule.

In some embodiments, the protein of interest is a therapeutic protein. In some embodiments, the therapeutic protein is a therapeutic antibody, enzyme, hormone, or growth factor. In some embodiments, aspects of the disclosure are useful to covalently attach a therapeutic protein to one or more polymers, for example, to improve a biophysical or pharmacokinetic property of the therapeutic protein.

In some embodiments, the protein of interest is used as an affinity protein or tag capable of binding to a molecule of interest. In some embodiments, aspects of the disclosure are useful to covalently immobilize an affinity protein to a solid support so that it can be used to purify or characterize one or more molecules (e.g., one or more molecules in a biological or assay sample) that interact with (e.g., bind to) the affinity protein. In some embodiments, aspects of the application relate to methods and systems for generating immobilized functional GST-fusion protein columns that are useful for affinity enrichment or purification.

In some embodiments, the first binding partner is contacted with a crosslinking agent (e.g., a zero-length crosslinking agent) prior to contact with the second binding partner. In some embodiments, the first binding partner is covalently attached to a solid support. In some embodiments, the first binding partner is covalently attached to a further molecule that is non-covalently bound to a solid support.

In some embodiments, a wash step is conducted immediately following contacting a first binding partner with a crosslinking agent (e.g., a zero length crosslinker). In some embodiments, a second binding partner is contacted with the immobilized first binding partner that had been previously contacted with a crosslinking agent (e.g., a zero length crosslinker). In some embodiments, the second binding partner is contacted with the first binding partner immediately following a wash step (e.g., to remove unbound crosslinking agent). In some embodiments, the crosslinking agent (e.g., a zero length crosslinker) connected to the first binding partner covalently links the first binding partner to the second binding partner after the two binding partners bind to each other.

In some embodiments, the second binding partner is attached (e.g., covalently) to an affinity protein. Accordingly, in some embodiments, an affinity protein can be immobilized to a solid support via a first binding partner that is covalently connected to a second binding partner. In some embodiments, an affinity protein comprises a receptor. In some embodiments, an affinity protein comprises an Fc gamma receptor IIIa (FcγRIIIa), Fc gamma receptor IIa, or a fragment thereof.

In some embodiments, a solid support comprises a synthetic resin. In some embodiments, a solid support comprises sepharose, agarose, silica, or polystyrene-divinyl-benzene. In some embodiments, a solid support comprises sepharose beads. In some embodiments, a solid support is arranged in a column. In some embodiments, a column is a reversed phase column (e.g., a C1, C4, C8, C18, C30, a phenyl reversed, an alkyl reversed, or other reversed phase column).

In some embodiments, the first binding partner comprises glutathione (GSH) and the second binding partner comprises glutathione S-transferase (GST). In some embodiments, the first binding partner comprises glutathione (GSH) and the second binding partner comprises structural maintenance of chromosomes 1 (SMC1). In some embodiments, the first binding partner comprises glutathione (GSH) and the second binding partner comprises RalA Binding Protein 1 (RALBP1). In some embodiments, the first binding partner comprises streptavidin and the second binding partner comprises biotin.

In some embodiments, a first binding partner and a second binding partner are covalently crosslinked. In some embodiments, a first binding partner and a second binding partner are covalently crosslinked via a crosslinking agent. In some embodiments, a crosslinking agent links a primary amine to a primary amine. In some embodiments, a crosslinking agent comprises a N-hydroxysuccinimide (NHS) reactive group. In some embodiments, a crosslinking agent is disuccinimidyl suberate (DSS). In some embodiments, a crosslinking agent is a zero-length crosslinking agent. In some embodiments, a zero-length crosslinking agent links a carboxylic acid to a primary amine. In some embodiments, a zero-length crosslinking agent is 1-ethyl-3-(dimethylaminopropyl) carbodiimide (EDC). In some embodiments, a zero-length crosslinking agent is dicyclohexylcarbodiimide (DCC).

In some embodiments, an affinity resin comprises a solid support material bound to glutathione (GSH) and an affinity protein bound to glutathione S-transferase (GST), wherein the GSH and GST are covalently linked by an amide bond. In some embodiments, an affinity chromatographic device comprises a solid support material bound to glutathione (GSH) and an affinity protein bound to glutathione S-transferase (GST), wherein the GSH and GST are covalently linked by an amide bond. In some embodiments, an affinity chromatographic device comprises a chromatographic column containing a solid support material bound to GSH and an affinity protein bound to GST.

In some embodiments, one or more residues of GST is covalently linked to GSH. In some embodiments, the covalent linkage between GST and GSH occurs at the binding interface (e.g., between GSH and one or more residues of the binding site of GST). In some embodiments, one or more residues of GST (e.g., one or more Lysines and/or Glutamines) is/are covalently linked to GSH by an amide bond. In some embodiments, Lysine 44 and/or Glutamine 51 of GST is/are covalently linked to GSH by an amide bond. In some embodiments, Lysine 44 of GST is covalently linked to GSH by an amide bond. In some embodiments, Glutamine 51 of GST is covalently linked to GSH by an amide bond. In some embodiments, Lysine 45 of GST from Schistosoma japonicum (UniProt entry GST26_SCHJA amino acids 1-218), or an equivalent amino acid position in a GST from a different species, is covalently linked to GSH by an amide bond.

In some embodiments, a method for purifying a protein includes contacting a sample comprising the protein to an affinity resin or an affinity chromatographic device that is produced as described in this application. In some embodiments, a wash buffer is applied to the affinity resin or affinity chromatographic device immediately after applying the sample. In some embodiments, a protein to be purified is eluted from the affinity resin or affinity chromatographic device after washing with the wash buffer.

These and other aspects are described in more detail below.

### BRIEF DESCRIPTION OF THE DRAWINGS

FIG. 1 illustrates a non-limiting protocol for connecting a protein of interest to a substrate.

FIGS. 2A-2C illustrate non-limiting examples of a protein of interest connected to different substrates.

FIG. 3 illustrates an example of binding and elution of IgG1 from GST-FcγRIIIa attached to GSH-Sepharose. Non-reducing 4-12% Bis Tris SDS-PAGE of (1) GST-FcγRIIIa standard, (2) purified IgG1 standard, and (3) low pH eluate (50 mM citrate 100 mM NaCl, pH 4.2 and 100 M glycine pH 3.0) from IgG1 binding to non-covalently attached GST-FcγRIIIa to GSH-Sepharose. 2 μg of protein was loaded onto each lane. Molecular weight markers are listed.

FIG. 4 illustrates an example of binding quantification of Mab1 to GST-FcγRIIIa conjugated to NHS-Sepharose. Bar graphs show the % binding of Mab1 and deglycosylated Mab1 GST-FcγRIIIa-NHS-Sepharose column. Percent bound is calculated by dividing the A280 absorbance of the eluate by the A280 absorbance of the starting material.

FIGS. 5A and 5B illustrate a non-limiting embodiment of EDC-mediated crosslinking of GSH to potential amino acids within the GST substrate. (FIG. 5A) Scheme of localized EDC-crosslinking procedure of GSH-Sepharose to GST-FcγRIIIa. (FIG. 5B) Simple schematic of EDC-activated-GSH inside the GST substrate pocket based on the GSH GST crystal structure and illustrated using ChemBioDraw. The proximity of GSH relative to Ser65, Lys44, Trp38, Gln64 and Gln51 shown in this schematic were based on the interactions generated by a PoseView image (PDB 1AQW).^{10, 12}

FIGS. 6A-6C illustrate a non-limiting embodiment of antibody binding to EDC-mediated crosslinked GST-FcγRIIIa to GSH-Sepharose. (FIG. 6A) Quantification of the binding of Mab1 to GST-FcγRIIIa conjugated using global crosslinking or localized crosslinking. Bar graphs show the % binding of Mab1 to the respective GST-FcγRIIIa columns. Percent bound is calculated by dividing the A280 absorbance of the eluate by the A280 absorbance of the starting material. (FIG. 6B) Non-reducing 4-12% Bis Tris SDS-PAGE of (1) binding and low pH elution of IgG1 from GST-FcγRIIIa noncovalently bound to GSH-Sepharose and (2) binding and low pH elution of IgG1 from GST-FcγRIIIa covalently attached to GSH-Sepharose by localized EDC crosslinking. 2 μg of protein was loaded onto each lane. Molecular weight markers are listed. Note that lane 1 in this figure is the same as 3 in FIG. 3. (FIG. 6C) Fluorescent images of GST-FcγRIIIa crosslinked to GSH-Sepharose in the absence (− EDC) or presence (+ EDC) of EDC stained with FITC-conjugated anti-CD16 (FcγRIIIa) antibody. The image insets are the phase contrast images of the fluorescent images. According to the manufacturer, the mean particle size of the beads is 90 μm.

FIG. 7 illustrates an example of quantification of % binding of Mab1 and Degly-Mab1 and Mab2 and Degly-Mab2 to covalently attached GST-FcγRIIIa to GSH-Sepharose. % bound is calculated by dividing the A280 absorbance of the eluate by the A280 absorbance of the starting material.

FIGS. 8A-8C illustrate an example of enrichment of nonfucosylated IgG1 using localized EDC crosslinked GST-FcγRIIIa Sepharose Chromatography. (FIG. 8A) Typical Feed and Elution profile of IgG1 from GST-FcγRIIIa column. Arrows point to unbound, low pH elution enriched peak 1 and low pH elution enriched peak 2. (FIG. 8B) Percent total nonfucosylation of IgG1 in each sample as measured by the 2-AB N-glycan analysis assay. (FIG. 8C) Results of AlphaScreen-based FcγRIIIa competitive binding assay comparing the enriched peak 1 sample and the unbound sample to the starting material sample. Starting material: open circles; Unbound: open squares; Enriched: open triangles.

FIGS. 9A and 9B illustrates a non-limiting embodiment of a procedure for attaching a protein to one or more polymers, for example, to improve a biophysical or pharmacokinetic property of the therapeutic protein. FIG. 9A illustrates a PEGylation procedure of GST-FcγRIIIa to GSH-PEG. FIG. 9B illustrates gel electrophoresis analysis of GST-FcγRIIIa coupled to GSH-PEG.

### DETAILED DESCRIPTION OF DISCLOSURE

Aspects of the disclosure provide methods and compositions for attaching a protein of interest to a solid support, a polymer, or other molecule. In some embodiments, techniques described in this application can be implemented using simple bifunctional cross-linkers to attach proteins to a substrate without introducing unwanted cross-links within the protein that could impact its structure and/or function. Techniques described in this application can be used to attach a protein to a substrate using a predictable and precise cross-linking technique that can be applied to different proteins of interest and different solid supports, polymers, or other molecules.

In some embodiments, aspects of the disclosure are useful to attach a protein to a solid support that can then be used as an affinity purification product to analyze and/or isolate one or more molecules that interact with the protein. In some embodiments, aspects of the disclosure are useful to attach a polymer or other molecule to a protein, for example to reduce the immunogenicity, increase the stability, or improve one or more other properties of the protein. In some embodiments, techniques of this application can be used to attach one or more polymers (e.g., polyethylene glycol) or other molecules to a therapeutic protein in order to improve one or more pharmacokinetic properties of the therapeutic protein.

In some embodiments, one or more modifications are made to a) a protein of interest, and b) a solid support, polymer, or other molecule so that they can be covalently attached in a predictable and controlled manner that does not involve unwanted (e.g., random or excessive) cross-linking of the protein of interest (e.g., between amino acids within the protein and/or between the protein and the solid support, polymer, or other molecule). Accordingly, in some aspects, the present application provides a generic method for attaching a protein of interest to a solid support, a polymer, or other molecule without altering or modifying the protein of interest in a way that would significantly impact its structure or function.

Covalent bonding such as N-hydroxysuccinimide (NHS) chemistry to crosslink the primary amine of lysines and 1-ethyl-3-(dimethylaminopropyl) carbodiimide (EDC) chemistry to crosslink the carboxylic acid groups from the aspartic or glutamic acids are common chemistries used to immobilize proteins to solid supports. However, crosslinking to the R groups of proteins via these highly reactive covalent chemistries can produce unwanted modifications of amino acids co-located near sites of biological function. In some cases, these modifications affect and alter the structure and/or function of the crosslinked protein (e.g., the binding and specificity of an immobilized receptor to its ligand).

In contrast, techniques described in this application can be used to cross-link a protein of interest to another moiety (e.g., a solid support, a polymer, or other molecule) without introducing unwanted crosslinks into the protein of interest.

In some embodiments, a solid support, polymer, or other molecule is attached (e.g., chemically) to a first member of a pair of molecules that bind to each other with high affinity (a first binding partner). The protein of interest is attached (e.g., chemically or synthesized as a fusion protein) to a second member of a pair of molecules that bind to each other with high affinity (a second binding partner). The attached molecules are contacted to each other under conditions that allow the first and second binding partners to bind to each other, and the associated binding partners are covalently linked using a cross-linker that is selected to connect a reactive group (e.g., an amine or a carboxyl group) in the first binding partner to a reactive group (e.g., an amine or carboxyl group) in the second binding partner. In some embodiments, the cross-linking is performed using methods that do not cross-link any reactive groups within the protein of interest.

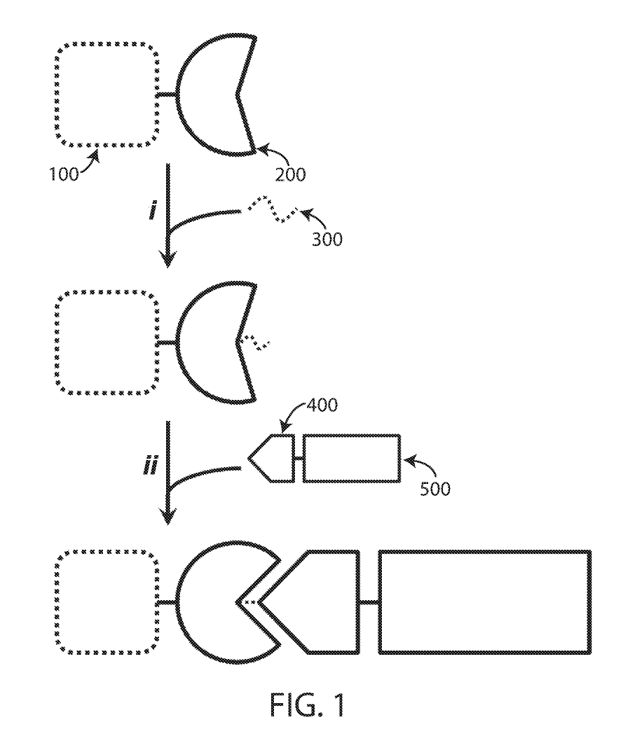

FIG. 1 illustrates a non-limiting embodiment of a method for connecting a substrate to a protein of interest using a crosslinking agent without introducing unwanted crosslinks into the protein of interest. In step (i), a crosslinking agent (300) is contacted to a first binding partner (200) that is attached to substrate (100). In some embodiments, step (i) is performed under a first set of reaction conditions that are suitable for the crosslinking agent to form a covalent bond with a reactive group in the first binding partner thereby producing a primed first binding partner (a first binding partner that is covalently linked to a crosslinking agent that is capable of reacting with a reactive group in a second molecule, for example in a second binding partner that binds to the first binding partner). In step (ii), the primed first binding partner is contacted to a second binding partner (400) that is attached to a protein of interest (500). In some embodiments, step (ii) is performed under a second set of reaction conditions that are suitable for a) the first and second binding partners to bind to each other, and b) for the crosslinking agent attached to the first binding partner to form a covalent bond with a reactive group in the second binding partner, thereby connecting the protein of interest to the substrate via a covalent link between the first and second binding partners. In some embodiments, depending on the type of crosslinker being used, the first and second reaction conditions are the same. In some embodiments, depending on the type of crosslinker being used, the first and second reaction conditions are different.

In some embodiments, the method is performed under conditions that avoid or reduce the presence of free crosslinking agent in step (ii) in order to avoid or reduce unwanted cross-linking of the protein of interest. In some embodiments, free crosslinking agent (crosslinking agent that is not covalently linked to the first binding partner) is removed (e.g., via a wash step, chromatography, or other procedure) after step (i) and before step (ii). In some embodiments, step (i) is performed under conditions (e.g., using equimolar amounts of first binding partner and crosslinking agent or excess amounts of first binding partner relative to crosslinking agent) to promote reaction of all or more of the crosslinking agent with the first binding partner and avoid the presence of significant (if any) amounts of unbound crosslinking agent after step (i).

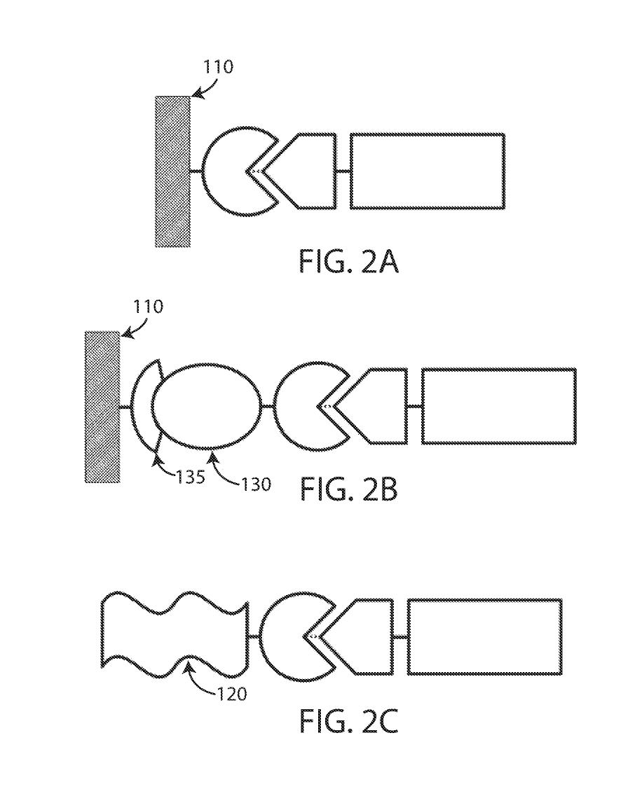

FIGS. 2A-2C illustrate non-limiting embodiments of different substrates that can be attached to a protein of interest as described in this application. FIG. 2A illustrates an embodiment where the protein of interest is connected to solid support (110) via a first binding partner that is covalently attached to the solid support (110). FIG. 2B illustrates an embodiment where the protein of interest is connected to solid support (110) via a first binding partner that is non-covalently attached to the solid support (110). The first binding partner is covalently attached to binding molecule (130) that binds specifically (but non-covalently) to binding molecule (135). Binding molecule (135) is covalently attached to the solid support (110). FIG. 2C illustrates an embodiment where the protein of interest is attached to a polymer or other molecule (120) via a first binding partner that is covalently attached to the polymer or other molecule (120). Although not illustrated, it should be appreciated that other configurations can be prepared using methods described in this application. For example, in some embodiments the first binding partner can be non-covalently attached to the polymer or other molecule. Similarly, in some embodiments the second binding partner is covalently attached to the protein of interest and in some embodiments the second binding partner is non-covalently attached to the protein of interest.

In some embodiments, a protein of interest can be an affinity protein that is attached to a solid support in order to purify or characterize molecules that bind to the affinity protein. In some embodiments, an affinity protein attached to a solid support can be used to purify or enrich one or more target molecules that bind to the affinity protein and that are present in a biological or other sample. In some embodiments, an affinity protein attached to a solid support can be used to screen for novel binding partners that bind to the affinity protein.

In some embodiments, suitable binding partners and crosslinking agents are selected based on the presence of one or more reactive groups in the binding partners (e.g., one or more amino acids having a reactive amine or carboxyl group), the relative proximity of a reactive group in the first binding partner to a reactive group in the second binding partner when the binding partners are bound to each other, and/or the length of the crosslinking agent (e.g., the distance between the functional crosslinking groups in the crosslinking agent). Other factors to consider when selecting binding partners include the ability to attach them (e.g., covalently) to the substrate and/or protein of interest, and/or their biocompatibility or other physiological properties (for example if they are going to be attached to a therapeutic protein). Similarly, one or more physical, biological, and/or physiological properties of a crosslinking agent can be considered when selecting one or more crosslinking agents to use as described herein.

In some embodiments, methods of the disclosure include the identification of reactive groups (e.g., amine, carboxyl, sulfhydryl groups (e.g., on cysteines) on proteins, and carbonyl groups on sugar residues of glycoproteins) at the binding interface of two non-covalent binding partners. In some aspects, identification of reactive groups at the binding interface allows for the selection of a suitable type of crosslinker reactivity. Non-limiting embodiments of types of crosslinker reactivity include amine to amine crosslinking, carboxyl to carboxyl crosslinking, and amine to carboxyl crosslinking. For example, in some embodiments, a suitable type of crosslinker reactivity could include the use of an amine to carboxyl crosslinker to crosslink binding partners having a binding interface that contains one or more amino acids with an amine side chain (or an N-terminal amine) on one binding partner and one or more amino acids with a carboxyl side chain (or a C-terminal carboxyl) on the other binding partner. It also should be appreciated that the distance at the binding interface between the amine reactive group(s) on one binding partner and the carboxyl reactive group(s) on the other binding partner should be considered when selecting the length of a suitable crosslinking agent. Similar considerations should be evaluated when using amine to amine and/or carboxyl to carboxyl crosslinking agents.

In some embodiments, the first binding partner is linked to a solid support and methods described herein are useful to prepare affinity material (e.g., affinity resins or other chromatography material) wherein the affinity tag (e.g., the affinity protein of interest) is covalently connected to the solid support (e.g., via covalently cross-linked binding partners). In some embodiments, affinity material described in this application has several advantages over current material, including i) the affinity protein is covalently attached to the solid support (as opposed to non-covalently attached, for example, using binding partners that are not cross-linked), and ii) the affinity protein is attached via crosslinking of the binding partners as opposed to via one or more random crosslinks of the protein directly to the solid support. The resulting affinity material has the dual benefit of reduced leaching and ability to withstand high stringency binding and wash conditions while retaining a desired structure and function of the affinity protein. According to aspects of the present disclosure, current systems that involve non-covalent binding of affinity proteins to solid supports do not allow for the purification of high affinity molecules because they do not allow for high stringency buffer conditions to be used.

The conceptual approach to affinity purification involves the immobilization of an affinity protein, wherein the affinity protein is capable of binding to a molecule of interest. A sample containing the molecule of interest is passed through a system comprising the immobilized affinity protein. After the immobilized affinity protein binds to the molecule of interest, the molecule of interest is eluted. A successful purification relies upon utilizing proper buffer conditions at each step. For example, buffer conditions used during a binding step can be adapted to promote binding of an affinity protein to a molecule of interest, buffer conditions used during a wash step can be adapted to remove contaminants, and buffer conditions used in an elution step can be adapted to disrupt the interaction between an affinity protein and a molecule of interest. The buffer conditions used in an elution step can be stringent enough to disrupt the binding between an affinity protein and a molecule of interest without the conditions being so stringent that they disrupt the immobilization of the affinity protein (e.g., if it is not covalently connected to the solid support) or are detrimental to the molecule of interest.

Current experimental techniques for affinity protein immobilization may involve reversible binding between a solid support and a molecular tag fused to an affinity protein. Exemplary current immobilization techniques include histidine and glutathione S-transferase molecular tags that can reversibly bind to Immobilized Metal Ion Chromatography resin (IMAC) and glutathione (GSH), respectively. While these non-covalent interactions are useful to bind and enrich for weak binding interactions, they may not be strong enough to withstand the high stringency elution buffers required to disrupt strong interactions such as the binding of FcγRIIIa to nonfucosylated IgG1s.⁶ In some aspects, a high stringency elution buffer comprises high salt concentrations that can result in protein denaturation. In some aspects, GST is unable to bind GSH under denaturing conditions (e.g., high salt concentrations), thereby disrupting immobilization of the affinity protein and contaminating the resulting elution product. In some aspects, a high stringency elution buffer comprises a low pH. In some aspects, one or more protonation events at a low pH can render histidine tags incapable of chelating metal ions, wherein immobilization of the affinity protein would be disrupted and the final elution product impure.

In contrast affinity proteins that are immobilized using methods described in the current application are capable of withstanding high stringency binding, wash, and/or elution solutions.

Accordingly, in some embodiments, aspects of the present disclosure relate to methods for immobilizing an affinity protein. An “affinity protein” can be any protein that forms a reversible interaction with a specified molecule. Exemplary affinity proteins include, without limitation, antibodies, antigens, immunoglobulins, hormones, growth factors, DNA-binding proteins, transport proteins, chaperone proteins, plasma proteins, enzymes, and receptors. In some aspects, affinity protein receptors comprise Fc gamma receptor IIIa (FcγRIIIa), Fc gamma receptor IIa or a fragment thereof. In some aspects, an affinity protein can be used to isolate a desired molecule from a complex mixture (e.g., a biological sample). In some aspects, an affinity protein can form non-covalent interactions with a desired molecule, wherein the interactions are characterized by a dissociation constant (K_{d}) on the order of 10⁻¹⁵ M to 10⁻⁷ M, inclusive. For example, an affinity protein and a desired molecule interact with a K_{d} on the order 10⁻⁵ M to 10⁻¹³ M, 10⁻¹⁴ M to 10⁻¹² M, 10⁻¹³ M to 10⁻¹ M, 10⁻¹² M to 10⁻¹⁰ M, 10⁻¹ M to 10⁻⁹ M, 10⁻¹⁰ M to 10⁻⁸ M, or 10⁻⁹ M to 10⁻⁷ M. In some aspects, an affinity protein and a desired molecule interact with a K_{d} on the order of 10⁻¹⁵ M. In some aspects, an affinity protein and a desired molecule interact with a K_{d} of 10⁻¹⁴ M. In some aspects, an affinity protein and a desired molecule interact with a K_{d} of 10⁻¹³ M. In some aspects, an affinity protein and a desired molecule interact with a K_{d} of 10⁻¹² M.

In some embodiments, methods of the present disclosure are useful for isolating a desired molecule that displays a high affinity for an immobilized affinity protein, wherein the high affinity interaction necessitates stringent buffer conditions (e.g., low pH, high salt concentrations) that could interfere with the immobilization of the affinity protein or alter the ability of the affinity protein to bind a desired molecule. In some aspects, stringent buffer conditions could interfere with the immobilization or functionality of an affinity protein during a binding step. In some aspects, stringent buffer conditions could interfere with the immobilization or functionality of an affinity protein during a wash step. In some aspects, stringent buffer conditions could interfere with the immobilization or functionality of an affinity protein during an elution step. An exemplary high affinity interaction is that of the protein binding pair barnase and barstar, the binding of which is described by a K_{d} of 10⁻¹⁴ M. According to the method of the present disclosure, isolation of the desired molecule (e.g., barstar) from the high affinity complex is facilitated by the covalent immobilization of the designated affinity protein (e.g., barnase) due to the inability of the stringent buffer conditions to disrupt the covalent immobilization.

Non-limiting examples of high stringency binding and/or wash conditions include low pH (e.g., pH from 1.0 to 2.0, 1.5 to 2.5, 2.0 to 3.0, 2.5 to 3.5, 3.0 to 4.0, 3.5 to 4.5, 4.0 to 5.0, 4.5 to 5.5, or 5.0 to 6.0), high pH (e.g., pH from 8 to 8.5, 8.2 to 8.7, or 8.5 to 9.0), high salt, high temperature, and/or the presence of one or more detergents, chaotropic agents, solvents (for example, organic solvents, e.g., acetonitrile), or other agents that can provide a high stringency environment. In some embodiments, high stringency binding and/or wash conditions can include the use of an inert solvent (e.g., a solvent that is non-reactive with the amino acids of a protein or the chemical moieties of a crosslinking agent). In some embodiments, the inert solvent is an organic solvent. In some embodiments, the organic solvent is acetonitrile.

In some aspects, the present disclosure relates to a method for immobilizing an affinity protein to a solid support. A “solid support” is any stationary phase of a chromatographic separation that can be functionalized to interact with an affinity protein. Exemplary solid supports include, without limitation, synthetic resin, polysaccharide compounds, sepharose, agarose, silica, activated alumina, kieselguhr, poly(vinyl chloride), and polystyrene-divinyl-benzene.

In some aspects, the present disclosure relates to a method for immobilizing an affinity protein to a solid support via the covalent chemical linkage of a first binding partner conjugated to said solid support and a second binding partner conjugated to said affinity protein. In some aspects, the first binding partner can be immediately conjugated to a solid support. In some aspects, the first binding partner can be conjugated to a target molecule that is non-covalently bound to a solid support. A “first binding partner” and “second binding partner” can comprise a pair of molecules that form non-covalent interactions. In some aspects, a first and second binding partner can comprise a small molecule and a protein, respectively. In some aspects, the first and second binding partners can comprise glutathione (GSH) and glutathione S-transferase (GST), respectively. GST from any organism or genetic location can be used. GST gene structure and function is known in the art (see e.g., Picket et al. Glutathione S-Transferases: Gene Structure, Regulation, and Biological Function, Annual Review of Biochemistry, 58:743-764 (1989); Yamamoto et al. Sci. Rep. 6:30073 (2016)). In some embodiments, GST is GST class-mu (26 kDa) from Schistosoma japonicum (GenBank entry GST26_SCHJA, amino acids 1-218) (e.g., Coughlin et al. Analytical Biochemistry 505(2016)51-58). In some embodiments, GST is a class pi glutathione S-transferase from human placenta (GST hP1-1, e.g., Prade et al. Structure 5:1287-1295 (1997)). In some aspects, the first and second binding partners can comprise glutathione (GSH) and structural maintenance of chromosomes 1 (SMC1), respectively. SMC1 from any organism or genetic location can be used (e.g., Yazdi et al. Genes Dev 16(5):571-582 (2002)). In some aspects, the first and second binding partners can comprise glutathione (GSH) and RalA Binding Protein 1 (RALBP1), respectively. RALBP1 from any organism or genetic location can be used (e.g., Sharma et al. Arch Biochem Biophys. 391(2):171-9 (2001)). In some aspects, a first and second binding partner can comprise a protein and a small molecule, respectively. In some aspects, the first and second binding partners can comprise streptavidin and biotin, respectively. In some aspects, the first and second binding partners can comprise avidin and biotin, respectively. In some aspects, both a first and second binding partner can comprise proteins. In some aspects, the first and second binding partner can comprise barnase and barstar, respectively. In some aspects, the first and second binding partner can comprise barstar and barnase, respectively.

The terms “crosslinking agent” and “crosslinker” are used interchangeably herein, and refer to a molecule that mediates the covalent linkage of a first binding partner to a second binding partner. In some embodiments, the crosslinking agent mediates the covalent linkage of a first and second binding partner via two or more reactive moieties, wherein one or more reactive moiety of the crosslinking agent covalently attaches to a first binding partner and one or more reactive moiety of the crosslinking agent covalently attaches to a second binding partner. In some embodiments, the resulting covalent linkage between a first and second binding partner comprises the crosslinking agent. In some embodiments, a crosslinking agent is selected to link reactive groups in first and second binding partners that are separated by up to 5 angstroms. In some embodiments, a crosslinking agent is selected to link reactive groups in first and second binding partners that are separated by up to 10 angstroms. In some embodiments, a crosslinking agent is selected to link reactive groups in first and second binding partners that are separated by up to 15 angstroms, for example, 0 to 11 angstroms, or 15-30 angstroms. In some embodiments, the reactive groups are separated by about 0 angstroms, for example, with the use of a zero-length crosslinker (e.g., EDC). In some embodiments, the reactive groups are separated by about 11 angstroms (e.g., with the use DSS). In some embodiments, a crosslinking agent conjugates a primary amine with a primary amine. In some embodiments, a crosslinking agent comprises a N-hydroxysuccinimide (NHS) reactive group. Exemplary crosslinkers that conjugate a primary amine with a primary amine via NHS reactivity include, without limitation, disuccinimidyl suberate (DSS), disuccinimidyl glutarate (DSG), bis(sulfosuccinimidyl)suberate (BS3), tris-(succinimidyl)aminotriacetate (TSAT), dithiobis(succinimidyl propionate) (DSP), 3,3′-dithiobis(sulfosuccinimidyl propionate) (DTSSP), disuccinimidyl tartrate (DST), and bis(2-(succinimidooxycarbonyloxy)ethyl)sulfone (BSOCOES). In some embodiments, a crosslinking agent comprises an imidoester reactive group. Exemplary crosslinkers that conjugate a primary amine with a primary amine via imidoester reactivity include, without limitation, dimethyl adipimidate (DMA), dimethyl pimelimidate (DMP), dimethyl suberimidate (DMS), and dimethyl 3,3′-dithiobispropionimidate (DTBP). In some embodiments, a crosslinking agent comprises a difluoro reactive group. Exemplary crosslinkers that conjugate a primary amine with a primary amine via difluoro reactivity include, without limitation, 1,5-difluoro-2,4-dinitrobenzene. Additional exemplary cross-linkers include PEGylated bis(sulfosuccinimidyl)suberate (BS(PEG)5), PEGylated bis(sulfosuccinimidyl)suberate (BS(PEG)9), ethylene glycol bis(succinimidyl succinate) (EGS), and ethylene glycol bis(sulfosuccinimidyl succinate) (Sulfo-EGS).

In some embodiments, the crosslinking agent mediates the covalent linkage of a first and second binding partner by activating a chemical group of a first binding partner, wherein activating the chemical group promotes the direct reactivity between the first binding partner and a second binding partner. In some embodiments, the resulting covalent linkage between a first and second binding partner does not comprise the crosslinking agent. In some embodiments, a crosslinking agent that is not retained in the resulting covalent linkage between a first and second binding partner is a zero-length crosslinker. A “zero-length crosslinker” is a molecule that mediates the covalent chemical conjugation of a first binding partner to a second binding partner without becoming part of the final crosslink between said binding partners. In some aspects, a zero-length crosslinker conjugates a carboxylic acid with a primary amine. Exemplary zero-length crosslinkers that conjugate a carboxylic acid with a primary amine include, without limitation, 1-ethyl-3-(dimethylaminopropyl) carbodiimide (EDC), carbonyldiimidazole (CDI), dicyclohexylcarbodiimide (DCC), N-hydroxysuccinamide (NHS), and N-hydroxysulfosuccinimide (Sulfo-NHS). In some embodiments, a zero-length crosslinker conjugates a carboxylic acid with a carboxylic acid. Exemplary zero-length crosslinkers that conjugate a carboxylic acid with a carboxylic acid include, without limitation, 1-ethyl-3-(dimethylaminopropyl) carbodiimide (EDC), carbonyldiimidazole (CDI), and other carbodiimide molecules.

In some embodiments, a zero-length crosslinker is contacted to the first binding partner under conditions that are suitable for (e.g., that promote) the initial covalent crosslinking reaction. In some embodiments, the initial covalent crosslinking reaction produces a primed first binding partner through the formation of a reactive intermediate between a carboxyl group of the first binding partner and an electrophilic atom of the zero-length cross linker (e.g., carbodiimide). In some embodiments, the reactive intermediate is a carboxylic ester with an activated leaving group. In some embodiments, the reactive intermediate is an O-acylisourea. A second binding partner can then be contacted to the modified first binding partner under conditions that are suitable for (e.g., that promote) both the final covalent crosslinking reaction and the specific interaction between the first binding partner and second binding partner. In some embodiments, the final covalent crosslinking reaction comprises a nucleophilic atom of the second binding partner forming a covalent bond with the activated carbon atom in the carboxyl group of the first binding partner. Upon formation of the covalent crosslink, the activating group provided by the zero-length crosslinker leaves as a by-product. In some embodiments, the resulting covalent crosslink is an amide bond. In some embodiments, the nucleophilic atom of the second binding partner is a primary amine. In some embodiments, the primary amine can be Lysine 44 or Glutamine 51. In some embodiments, the specific interaction between the first binding partner and second binding partner is non-covalent.

In some aspects, a first binding partner is conjugated to a crosslinker prior to conjugation of said first binding partner with a second binding partner. In some aspects, a wash step is performed prior to the conjugation of a first binding partner conjugated to a crosslinker with a second binding partner. In some embodiments, a wash step can include flowing buffer through a stationary phase, e.g., an immobilized first binding partner conjugated to a crosslinker, in order to remove excess, e.g., unbound, crosslinker. In some aspects, a buffer used in the wash step comprises the equilibration buffer used in the mobile phase. In some aspects, the buffer used in the wash step comprises a phosphate buffer. In some aspects, the buffer used in the wash step comprises Bis-Tris, CAPS, carbonate, HEPES, HEPPS, HEPPSO, MES, MOPS, MOPSO, phosphate, PIPES, POPSO, TAPS, TAPSO, TEA, TES, or Tris.

In some aspects, methods of the present disclosure relate to an affinity resin comprising a solid support bound to a first binding partner and an affinity protein bound to a second binding partner. The term “affinity resin” is used to encompass a suspension comprising a solid support that has been functionalized with an affinity protein. In some aspects, an affinity resin comprises a first binding partner and a second binding partner that are covalently linked. In some aspects, an affinity resin comprises a solid support bound to glutathione (GSH) and an affinity protein bound to glutathione S-transferase (GST), wherein the GSH and GST are covalently linked by an amide bond.

In some aspects, methods of the present disclosure relate to an affinity chromatographic device comprising a solid support bound to a first binding partner and an affinity protein bound to a second binding partner. The term “affinity chromatographic device” is used to encompass any vessel suitable for chromatography comprising a solid support that has been functionalized with an affinity protein. Exemplary vessels suitable for chromatography include, without limitation, columns, disks, tubes, and surfaces, and also include microfluidic channel. In some aspects, an affinity chromatographic device comprises a solid support bound to a first binding partner and an affinity protein bound to a second binding partner, wherein the first binding partner and the second binding partner are covalently linked. In some aspects, an affinity chromatographic device comprises a solid support bound to glutathione (GSH) and an affinity protein bound to glutathione S-transferase (GST), wherein the GSH and GST are covalently linked by an amide bond. In some aspects, an affinity chromatographic device comprises a chromatographic column containing a solid support bound to a first binding partner and an affinity protein bound to a second binding partner, wherein the first binding partner and the second binding partner are covalently linked. In some aspects, an affinity chromatographic device comprises a chromatographic column containing a solid support bound to GSH and an affinity protein bound to GST, wherein the GSH and GST are covalently linked by an amide bond.

In some aspects, the present disclosure relates to a method for purifying a protein. In some aspects, a protein is purified from a sample by contacting said sample with an affinity resin comprising an affinity protein immobilized to a solid support. In some aspects, a protein is purified from a sample by contacting said sample with an affinity chromatography separation device comprising a vessel containing an affinity protein immobilized to a solid support. In some aspects, a wash step is performed with the affinity chromatography separation device immediately following the addition of a sample comprising a protein to be purified. In some aspects, the protein to be purified is isolated by eluting said protein from the affinity resin. In some aspects, the protein to be purified is isolated by eluting said protein from the affinity chromatography separation device.

In some aspects, affinity material comprises a solid support bound to GSH and an affinity protein bound to GST. In some embodiments, one or more residues of GST is covalently linked to GSH. In some embodiments, the covalent linkage between GST and GSH occurs at the binding interface (e.g., between GSH and one or more residues of the binding site of GST). In some embodiments, one or more residues of GST is covalently linked to GSH by an amide bond. In some embodiments, one or more lysine or glutamine residues of GST is covalently linked to GSH by an amide bond. For example, in some embodiments, Lysine 44 and/or Glutamine 51 of GST (e.g., in human GST), or an equivalent amino acid position in a GST from a different species, is covalently linked to GSH by an amide bond. In some embodiments, Lysine 45 of GST from Schistosoma japonicum (UniProt entry GST26_SCHJA amino acids 1-218), or an equivalent amino acid position in a GST from a different species, is covalently linked to GSH by an amide bond.

Accordingly, in some embodiments, methods described in the present application can be used to localize the crosslinking of GST-fusion proteins such as GST-Fcγ receptors to GSH-sepharose. Such methods can use readily available reagents, such as EDC and GSH-sepharose, and results in immobilized GST-Fcγ receptors that are functional and specific. Fcγ receptor columns can be used to isolate and enrich for higher and lower affinity binding species of antibodies.

The separation of different molecules based on their respective binding to Fc receptors is known in the art, and includes antibodies with different degrees of fucosylation (e.g., Roche, US 2014-0255399) and polypeptide glycoforms (e.g., Zepteon, US 2013-0084648). Additionally, the use of an immobilized non-covalent complex including a neonatal Fc receptor and β-2-microglobulin as an affinity chromatography ligand is also known in the art (e.g., Roche, WO 2013/120929). Methods and compositions described herein provide improved affinity material for separating molecules based on their relative binding affinity to Fc receptors (or to other molecules of interest), for example without leaching of the affinity tag into the purified product, without loss of binding activity and specificity of the immobilized protein for its binding partner, and/or allowing for the separation of molecules that are characterized by high affinity binding to the affinity tag.

In some embodiments, a first binding partner can be covalently attached to a solid support using standard chemical reactions. In some embodiments, a first binding partner covalently attached to a solid support is commercially available (e.g., GST-sepharose). In some embodiments, a first binding partner can be coupled to a solid support (e.g., sepharose and/or agarose) via NHS or other suitable chemistry.

In some embodiments, a polymer is attached to a protein of interest as described herein in order to improve one or more physical or biological properties of the protein. Non-limiting examples of polymers include polyethylene glycol (PEG), carbohydrates, hydroxyethyl starch (HES), Dextran, Polysialic Acids (PSAs), Poly(2-ethyl 2-oxazoline) (PEOZ), and XTEN peptides.

In some embodiments, a first binding partner can be covalently attached to a polymer support using standard chemical reactions. In some embodiments, a binding partner covalently attached to a polymer is commercially available. In some embodiments, GSH-PEG is provided. In some embodiments, GSH-PEG comprises methoxypolyethylene glycol linked to monofunctional glutathione. In some embodiments, GSH-functionalized PEG was made by covalently linking PEG to the thiol group of GSH via a thiol ether bond.

In some embodiments, a protein of interest can be a therapeutic protein that is attached to a polymer (e.g., PEG) in order to reduce its immunogenicity or improve one or more of its physical or pharmacokinetic properties (e.g., bioavailability, stability, clearance, etc.). Non-limiting examples of therapeutic proteins include antibodies, enzymes, hormones, blood cascade factors, growth factors, receptors, and receptor binding polypeptides.

In some embodiments, a therapeutic protein is an antibody. In some embodiments, the antibody is STX-100, TYSABRI®, Daclizumab (DAC), BART, Tweak, or Anti-BDCA2. STX-100 is a humanized monoclonal antibody that targets integrin αvβ6. STX-100 exhibits significant anti-fibrotic activity in preclinical animal models of kidney, lung and liver disease. The FDA has previously granted orphan drug designation to STX-100 for chronic allograft nephropathy. TYSABRI® (Natalizumab) is a humanized monoclonal antibody against the cell adhesion molecule α4-integrin. Natalizumab is used in the treatment of multiple sclerosis and Crohn's disease. BART (BIIB037) is an anti-beta-amyloid human monoclonal antibody used as a treatment for Alzheimer's disease (AD). It is believed that BIIB037 binds to and eliminates toxic amyloid plaques that form in the brains of patients with AD, thereby potentially suppressing the progression of the disease. Anti-TWEAK is a humanized monoclonal antibody specific for TWEAK useful in the treatment of lupus nephritis (LN). Daclizumab (Zenapax®) is a therapeutic humanized monoclonal antibody used to prevent rejection in organ transplantation, especially in kidney transplants. Daclizumab works by binding to CD25, the alpha subunit of the IL-2 receptor of T cells.

In some embodiments, the antibody is: anti-LINGO, anti-LINGO-1, interferon (e.g., interferon beta 1a—AVONEX), Abciximab (REOPRO®), Adalimumab (HUMIRA®), Alemtuzumab (CAMPATH®), Basiliximab (SIMULECT®), Bevacizumab (AVASTIN®), Cetuximab (ERBITUX®), Certolizumab pegol (CIMZIA®), Daclizumab (ZENAPAX®), Eculizumab (SOLIRIS®), Efalizumab (RAPTIVA®), Gemtuzumab (MYLOTARG®), Ibritumomab tiuxetan (ZEVALIN®), Infliximab (REMICADE®), Muromonab-CD3 (ORTHOCLONE OKT3®), Natalizumab (TYSABRI®), Omalizumab (XOLAIR®), Palivizumab (SYNAGIS®), Panitumumab (VECTIBIX®), Ranibizumab (LUCENTIS®), Rituximab (RITUXAN®), Tositumomab (BEXXAR®), or Trastuzumab (HERCEPTIN®). In some embodiments, the antibody is Natalizumab (TYSABRI®).

In some embodiments, the antibody is Abagovomab, Abciximab, Actoxumab, Adalimumab, Adecatumumab, Afelimomab, Afutuzumab, Alacizumab pegol, ALD, Alemtuzumab, Alirocumab, Altumomab pentetate, Amatuximab, Anatumomab mafenatox, Anrukinzumab, Apolizumab, Arcitumomab, Aselizumab, Atinumab, Atlizumab, Atorolimumab, Bapineuzumab, Basiliximab, Bavituximab, Bectumomab, Belimumab, Benralizumab, Bertilimumab, Besilesomab, Bevacizumab, Bezlotoxumab, Biciromab, Bimagrumab, Bivatuzumab mertansine, Blinatumomab, Blosozumab, Brentuximab vedotin, Briakinumab, Brodalumab, Canakinumab, Cantuzumab mertansine, Cantuzumab ravtansine, Caplacizumab, Capromab pendetide, Carlumab, Catumaxomab, Cedelizumab, Certolizumab pegol, Cetuximab, Citatuzumab bogatox, Cixutumumab, Clazakizumab, Clenoliximab, Clivatuzumab tetraxetan, Conatumumab, Concizumab, Crenezumab, Dacetuzumab, Daclizumab, Dalotuzumab, Daratumumab, Demcizumab, Denosumab, Detumomab, Dorlimomab aritox, Drozitumab, Duligotumab, Dupilumab, Dusigitumab, Ecromeximab, Eculizumab, Edobacomab, Edrecolomab, Efalizumab, Efungumab, Eldelumab, Elotuzumab, Elsilimomab, Enavatuzumab, Enlimomab pegol, Enokizumab, Enoticumab, Ensituximab, Epitumomab cituxetan, Epratuzumab, Erlizumab, Ertumaxomab, Etaracizumab, Etrolizumab, Evolocumab, Exbivirumab, Fanolesomab, Faralimomab, Farletuzumab, Fasinumab, FBTA, Felvizumab, Fezakinumab, Ficlatuzumab, Figitumumab, Flanvotumab, Fontolizumab, Foralumab, Foravirumab, Fresolimumab, Fulranumab, Futuximab, Galiximab, Ganitumab, Gantenerumab, Gavilimomab, Gemtuzumab ozogamicin, Gevokizumab, Girentuximab, Glembatumumab vedotin, Golimumab, Gomiliximab, Guselkumab, Ibalizumab, Ibritumomab tiuxetan, Icrucumab, Igovomab, Imciromab, Imgatuzumab, Inclacumab, Indatuximab ravtansine, Infliximab, Intetumumab, Inolimomab, Inotuzumab ozogamicin, Ipilimumab, Iratumumab, Itolizumab, Ixekizumab, Keliximab, Labetuzumab, Lampalizumab, Lebrikizumab, Lemalesomab, Lerdelimumab, Lexatumumab, Libivirumab, Ligelizumab, Lintuzumab, Lirilumab, Lodelcizumab, Lorvotuzumab mertansine, Lucatumumab, Lumiliximab, Mapatumumab, Margetuximab, Maslimomab, Mavrilimumab, Matuzumab, Mepolizumab, Metelimumab, Milatuzumab, Minretumomab, Mitumomab, Mogamulizumab, Morolimumab, Motavizumab, Moxetumomab pasudotox, Muromonab-CD, Nacolomab tafenatox, Namilumab, Naptumomab estafenatox, Narnatumab, Natalizumab, Nebacumab, Necitumumab, Nerelimomab, Nesvacumab, Nimotuzumab, Nivolumab, Nofetumomab merpentan, Ocaratuzumab, Ocrelizumab, Odulimomab, Ofatumumab, Olaratumab, Olokizumab, Omalizumab, Onartuzumab, Oportuzumab monatox, Oregovomab, Orticumab, Otelixizumab, Oxelumab, Ozanezumab, Ozoralizumab, Pagibaximab, Palivizumab, Panitumumab, Panobacumab, Parsatuzumab, Pascolizumab, Pateclizumab, Patritumab, Pemtumomab, Perakizumab, Pertuzumab, Pexelizumab, Pidilizumab, Pinatuzumab vedotin, Pintumomab, Placulumab, Polatuzumab vedotin, Ponezumab, Priliximab, Pritoxaximab, Pritumumab, Quilizumab, Racotumomab, Radretumab, Rafivirumab, Ramucirumab, Ranibizumab, Raxibacumab, Regavirumab, Reslizumab, Rilotumumab, Rituximab, Robatumumab, Roledumab, Romosozumab, Rontalizumab, Rovelizumab, Ruplizumab, Samalizumab, Sarilumab, Satumomab pendetide, Secukinumab, Seribantumab, Setoxaximab, Sevirumab, Sibrotuzumab, Sifalimumab, Siltuximab, Simtuzumab, Siplizumab, Sirukumab, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tanezumab, Taplitumomab paptox, Tefibazumab, Telimomab aritox, Tenatumomab, Teneliximab, Teplizumab, Teprotumumab, TGN, Ticilimumab, Tildrakizumab, Tigatuzumab, TNX-, Tocilizumab, Toralizumab, Tositumomab, Tovetumab, Tralokinumab, Trastuzumab, TRBS, Tregalizumab, Tremelimumab, Tucotuzumab celmoleukin, Tuvirumab, Ublituximab, Urelumab, Urtoxazumab, Ustekinumab, Vantictumab, Vapaliximab, Vatelizumab, Vedolizumab, Veltuzumab, Vepalimomab, Vesencumab, Visilizumab, Volociximab, Vorsetuzumab mafodotin, Votumumab, Zalutumumab, Zanolimumab, Zatuximab, Ziralimumab or Zolimomab aritox.

In some embodiments, a therapeutic protein is a blood cascade protein. Blood cascade proteins are known in the art and include, but are not limited to, Factor VII, tissue factor, Factor IX, Factor X, Factor XI, Factor XII, Tissue factor pathway inhibitor, Factor V, prothrombin, thrombin, vonWillebrand Factor, kininigen, prekallikrien, kallikrein, fribronogen, fibrin, protein C, thrombomodulin, and antithrombin. In some embodiments, the blood cascade protein is Factor IX or Factor VIII. It should be appreciated that methods provided herein are also applicable for uses involving the production of versions of blood cascade proteins, including blood cascade proteins that are covalently bound to antibodies or antibody fragments, such as Fc. In some embodiments, the blood cascade protein is Factor IX-Fc (FIXFc) or Factor VIII-Fc (FVIIIFc). In some embodiments, one or more proteins of interest are hormones, regulatory proteins and/or neurotrophic factors. Neurotrophic factors are known in the art and include nerve growth factor (NGF), brain-derived neurotrophic factor (BDNF), neurotrophin-3 (NT-3), neurotrophin-4 (NT-4), members of the glial cell line-derived neurotrophic factor ligands (GDNF) and ciliary neurotrophic factor (CNTF). In some embodiments, the protein of interest is neublastin.

Non-limiting examples of other molecules that can be attached to a protein of interest using methods described herein include toxins. In some embodiments, a toxin can be conjugated to an antibody (e.g., by attaching the toxin to a first binding partner and fusing the antibody to a second binding partner, and crosslinking the first and second binding partners as described in this application). In some embodiments, the toxin is doxorubicin or mertansine.

The present invention is further illustrated by the following Examples, which in no way should be construed as further limiting. The entire contents of all of the references (including literature references, issued patents, published patent applications, and co-pending patent applications) cited throughout this application are hereby expressly and specifically incorporated by reference, in particular for the teaching that is referenced hereinabove (for example, but not limited to, for a teaching related to a cross-linker, an affinity protein, a solid support, or one or more binding partners (e.g., a ligand such as glutathione, or a protein binding partner such as GST), including any structures or sequences of any of the aforementioned).

### EXAMPLES

### Example 1: A Simple Enzyme-Substrate-Localized Conjugation Method to Generate Immobilized Functional GST-Fusion Protein Columns for Affinity Enrichment

Immobilized protein receptors and enzymes are useful purification tools for isolating or enriching different ligands and substrates based on their highly selective affinity. For example, Glutathione-S-Transferase (GST) is commonly covalently linked to protein sequences to serve as a molecular tag for binding to its substrate glutathione (GSH) which, can be easily linked to solid supports. One issue, however with this approach is that the high affinity interaction between receptors and their ligands requires harsh elution conditions such as low pH which can result in either leached receptor or generation of aggregates in the intended elution pool. Another issue with attaching receptors to solid supports is the inherent non-specific chemical conjugation of reactive groups such as using N-hydroxysuccinimide (NHS) chemistry that couples lysines to the solid support. Since NHS chemistry is not lysine site specific, this may result in the modification of those residues near the binding site(s) of the immobilized receptor that then affects its specificity for the intended ligand. In this study, a simple chemical conjugation procedure is presented that overcomes these limitations and results in immobilized GST-fusion proteins that are both functional and specific to the target without detectable leaching of the receptor from the column. Here, the affinity of GST for GSH was utilized to generate an enzyme-substrate site-specific crosslinking reaction: first, GSH-Sepharose was pre-activated with 1-ethyl-3-(dimethylaminopropyl) carbodiimide (EDC). Next, excess EDC was removed to prevent non-specific crosslinking. Finally, EDC-GSH-activated-Sepharose was incubated with GST-Fc gamma receptor IIIa (FcγRIIIa). The immobilized GST-FcγRIIIa specifically bound glycosylated IgG1s and the high affinity interaction was used to selectively isolate nonfucosylated IgG1s from weaker binding species such as fucosylated IgG1s. This technique can be used when modifications of critical amino acids leads to changes in activity.

In this example, a simple and straightforward chemical conjugation procedure is presented that resulted in immobilized GST-FcγRIIIa proteins that are both functional and specific for IgG1s differing in N-linked glycoforms. First, GSH-Sepharose is coupled to EDC. Next, excess EDC is removed to prevent non-specific crosslinking. Finally, this EDC-GSH-activated-Sepharose is incubated with GST-FcγRIIIa. The interactions of IgG1 to FcγRIIIa were studied to determine whether this general crosslinking procedure could purify highly specific forms of IgG1s (e.g., nonfucosylated) which are difficult to purify by conventional chromatography. Results showed that this enzyme-substrate EDC-immobilized GST-FcγRIIIa bound specifically to glycosylated IgG1s and was used to enrich for nonfucosylated IgG1s. The isolated nonfucosylated IgG1s were assayed in bioassays for functionality and were found to bind 6-fold tighter to FcγRIIIa. The described conjugation procedure is straightforward and fast (1-2 hours), has good capacity since the conjugated Fc receptors are functional, and can be carried out with common commercial reagents.

### Results and Discussion

### Immobilization of GST-FcγRIIIa and Testing for Function and Specificity

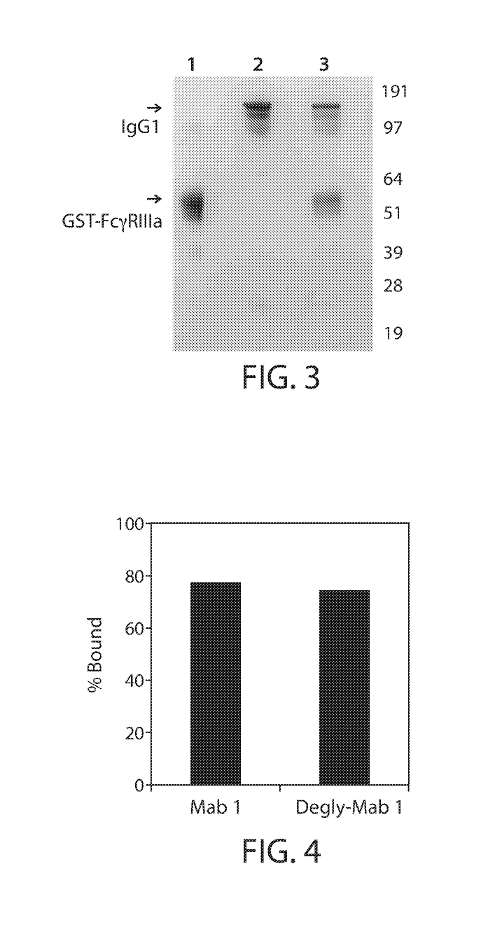

Fc gamma receptors display different affinity selectivity to different forms of IgG1s.^{6,8} FcγRIIIa binds tightly to nonfucosylated IgG1s and less tightly to fucosylated IgG1s (K_{d}˜7.2×10⁻⁹ and 3.0×10⁻⁷ respectively⁶). To isolate and enrich for different forms of IgG1s, several strategies were assessed to generate FcγRIIIa columns that could bind and distinguish low and high affinity interactions. First, GST-FcγRIIIa affinity columns were made using the noncovalent interaction between GST and commercially available GSH-Sepharose. Low pH elution conditions were used to elute bound forms of IgG1 such as nonfucosylated antibodies.³ However, the low pH buffer additionally disrupted the binding between the GST-FcγRIIIa and the GSH Sepharose resin itself, resulting in the simultaneous elution of a highly purified IgG1 antibody and GST-FcγRIIIa as visualized by non-reducing SDS-PAGE (FIG. 3, lane 3). This observation was consistent with the known binding affinities between GSH to GST and FcγRIIIa to IgG1s. The dissociation constant between GSH and GST is on the order of ˜10⁻⁴ whereas the dissociation between fucosylated IgG1s and FcγRIIIa is 1000 times stronger at ˜10⁻⁷.^{4,6} Furthermore, the dissociation constant between nonfucosylated IgG1s and FcγRIIIa is even stronger at ˜10⁻⁹.⁶ Thus, the attachment of FcγRIIIa to solid supports using noncovalent interactions was not suitable to study these strong interactions.

Thus, simple, off-the shelf chemical conjugation procedures were assessed to covalently immobilize GST-FcγRIIIa protein complexes onto solid supports to study these high and higher affinity interactions. First, covalently-immobilized FcγRIIIa affinity columns were made using commercial pre-packed N-hydroxysuccinimide (NHS)-Sepharose columns. The generation of this FcγRIIIa receptor column required a 30 minute incubation of GST-FcγRIIIa with NHS-Sepharose to allow for crosslinking of NHS with primary amines on GST-FcγRIIIa, followed by an overnight quenching of free NHS groups. To determine whether the NHS-conjugated GST-FcγRIIIa column retained its ability to bind its ligand, a highly purified IgG1 antibody (called Mab1), was loaded onto the column in a neutral pH buffer, and eluted with a low pH buffer.

This column binding experiment showed that NHS-conjugated GST-FcγRIIIa could bind to Mab1 (FIG. 4). To assess whether the NHS-conjugated GST-FcγRIIIa column retained its specificity for glycosylated antibodies, PNGase-F deglycosylated Mab1 (Degly-Mab1) was tested for its ability to bind the column. It has been demonstrated that removal of the glycan from antibodies abolishes IgG1 binding to FcγRIIIa.⁸ FIG. 4 showed that while NHS-FcγRIIIa column bound glycosylated Mab1, this column also bound Degly-Mab1, suggesting that the NHS reactive groups may have modified critical lysine groups within FcγRIIIa. FcγRIIIa has 12 lysine residues in addition to the N terminus and GST has 21 lysine groups that can react with NHS. Based on the crystal structure of IgG1 bound to FcγRIIIa, Lys158 and Lys117 contact IgG1 and may be critical for binding.⁶ A similar observation of nonspecific binding to IgG1s was observed with NHS-linked to the lower affinity Fc gamma receptor, GST-FcγRIIa (data not shown). Another possibility is that the nonspecific NHS chemistry may have cross-linked FcγRIIIa or GST into a non-suitable conformation. Nonetheless, this chemical procedure changed the functional specificity of FcγRIIIa allowing it to bind deglycosylated Mab1 and negating the ability for this column to distinguish and isolate weak or non-binding forms of IgG1s.

### Immobilization of GST-FcγRIIIa Using Enzyme-Substrate Specificity

Several studies describing the immobilization of enzymes to solid supports such as proteases and GST take advantage of the specific binding of these enzymes to their substrates to capture enzymes to solid supports.^{4,9} While these immobilization strategies led to site specific conjugation, these methods require more complex chemical functionalization, such as chemical derivatization of GSH with the photoreactive group benzophenone, which may not be commercially available.⁴

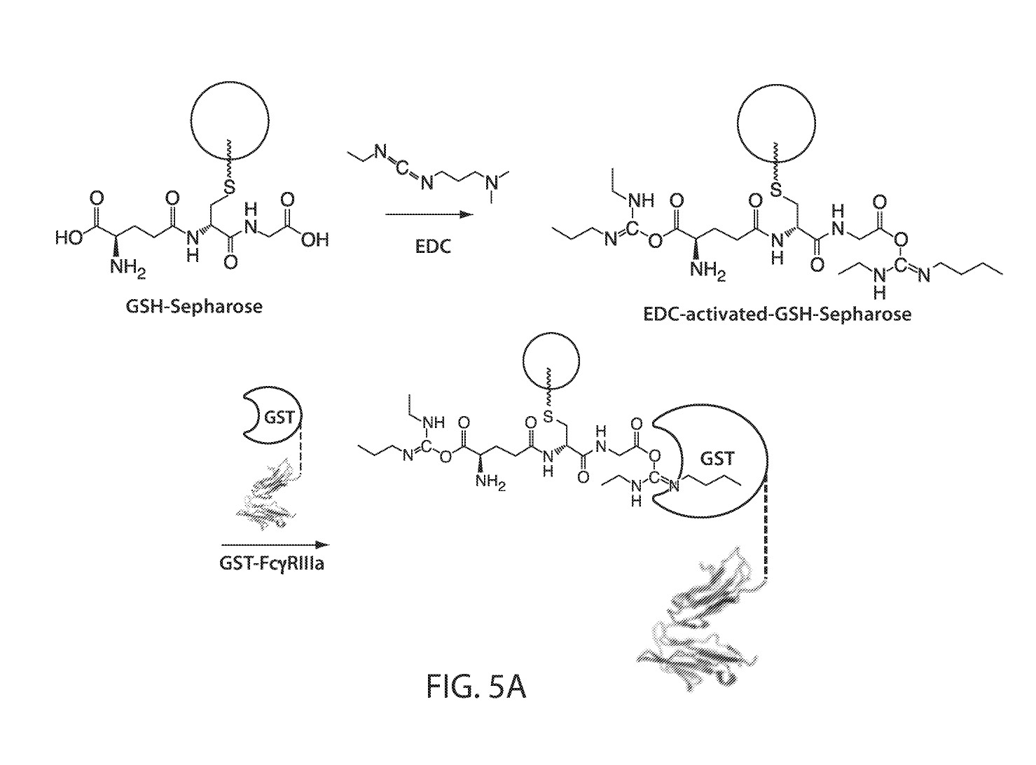

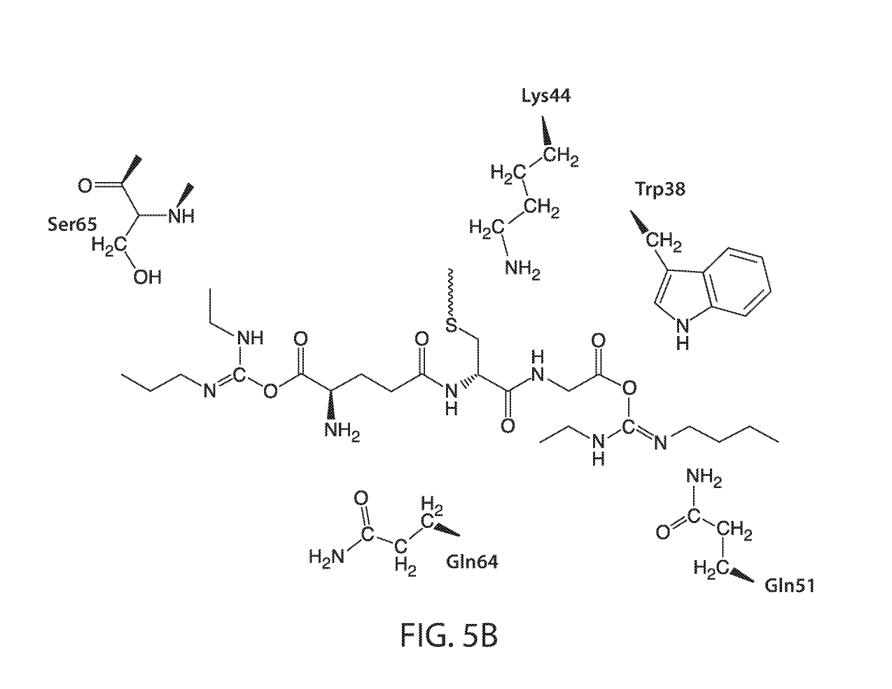

This study aimed to utilize a similar enzyme-substrate affinity to generate immobilized GST-FcγRIIIa receptors using simple straightforward chemical reactions that could be performed in aqueous buffers that would not affect protein stability or activity. The crystal structure of human GST bound to its substrate GSH shows several amine groups inside the GST pocket in close proximity to the terminal carboxylic acid groups on glutathione.¹⁰ In particular, and as shown in the simplified schematic in FIG. 5B, the primary amines from Lys44, Gln64 and Gln51 were within proximity of the carboxylic acids of glutathione. If these functional groups are very close, the zero length cross linker EDC could be used to covalently crosslink the C-terminal carboxylic acid of glutathione to one of these primary amine groups in GST versus non-specific cross-linking of the lysines as described earlier.

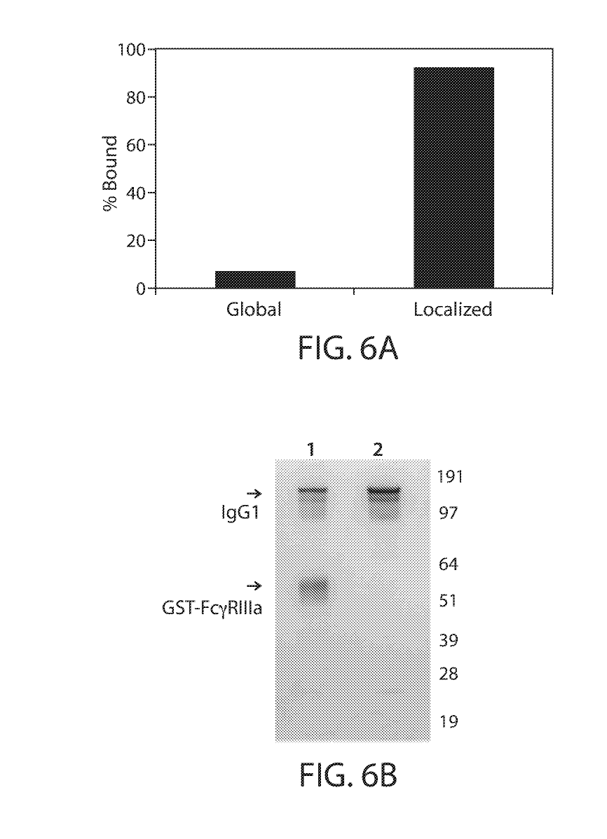

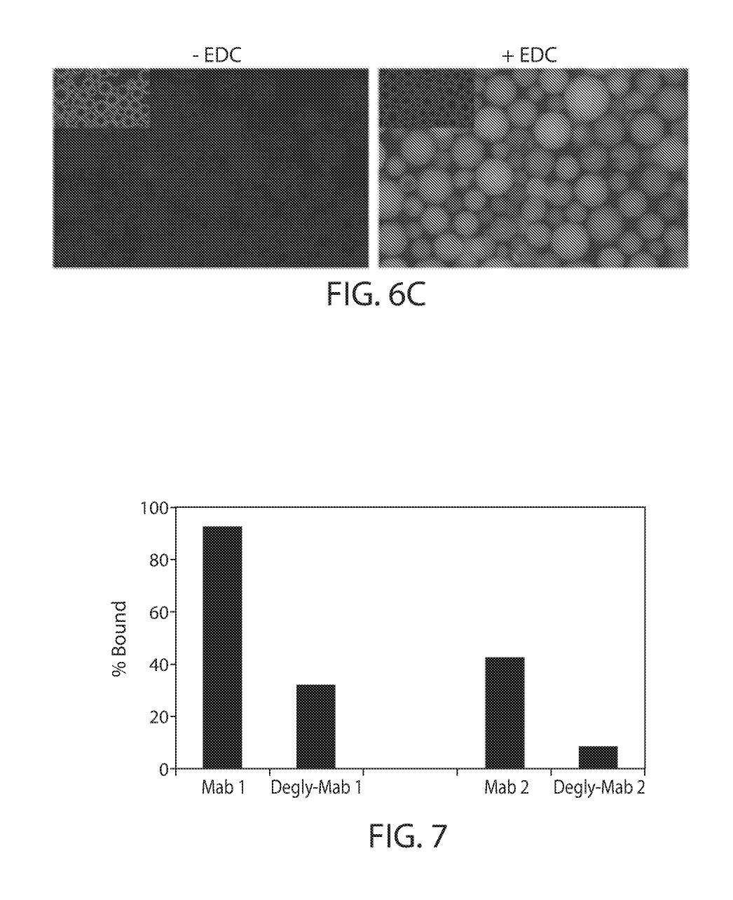

To test this hypothesis, GSH-Sepharose was pre-incubated with GST-FcγRIIIa to form a GSH-GST-FcγRIIIa non-covalent complex. Next, EDC was added to the preformed complex to crosslink the carboxylic acids of glutathione which may be in close proximity to several amine groups inside the GST enzyme pocket, such as Lys44 (FIG. 5B). This EDC crosslinking procedure resulted in immobilized GST-FcγRIIIa. To determine whether the EDC-conjugated GST-FcγRIIIa was still functional, Mab1 was tested for its ability to bind the column as was previously described in FIGS. 3 and 4. The results show that GST-FcγRIIIa columns made using this procedure resulted in less than 10% binding of Mab1 to the column (FIG. 6A, denoted as Global). There are 21 carboxylic acid residues in addition to the C terminus in FcγRIIIa that could potentially react with EDC and disrupt FcγRIIIa function.

To overcome this potential global over crosslinking of GSH to GST-FcγRIIIa, an alternative yet simple strategy was devised to more localize the crosslinking of GSH to the GST substrate pocket (FIG. 5B). GSH-Sepharose was first incubated with EDC to create an EDC-activated-GSH-Sepharose as shown in the scheme in FIG. 5A. Because EDC is highly labile, the EDC-activated-GSH-Sepharose was immediately incubated with GST-FcγRIIIa.¹¹ The immobilized GST-FcγRIIIa on this column could not be removed using low pH elution buffer as shown by non-reducing SDS-PAGE (FIG. 6B, lane 2). Approximately 1.5 to 3.0 mg/mL of GST-FcγRIIIa was conjugated to the resin.

In addition, the covalently bound GST-FcγRIIIa was tightly linked to the resin as it did not leach from the column from high pH, glutathione and SDS washes. Even after these washes, FcγRIIIa could be detected with a fluorescently labeled FITC-conjugated antibody to FcγRIIIa (FIG. 6C). In contrast, no FcγRIIIa was detected in the (−) EDC control beads (FIG. 6C).

To determine whether the GST-FcγRIIIa column generated using this localized approach was functional, Mab1 was tested for binding to the column. Unlike the global EDC crosslinking approach which resulted in FcγRIIIa that bound less than 10% of Mab1 (FIG. 7, Mab1), the GST-FcγRIIIa resin made using the EDC localized crosslinking approach was functional. As shown by SDS-PAGE electrophoresis (FIG. 6B, lane 2) and quantification of binding (FIG. 7, Mab1), over 90% Mab1 bound to the GST-FcγRIIIa resin. More importantly, this immobilized GST-FcγRIIIa bound 3-fold less Degly-Mab 1 than Mab1, supporting the hypothesis that crosslinking of GST-FcγRIIIa to GSH-Sepharose was localized to the GST-GSH site. This GST-FcγRIIIa resin also showed specificity to a different antibody, as Mab2 bound 5-fold more than Degly-Mab2 (FIG. 7). These data demonstrate that localized EDC crosslinking via the GST-GSH interaction results in FcγRIIIa that is both functional and specific for glycosylated antibodies.

To create an even simpler and more streamlined procedure, GST-FcγRIIIa affinity columns were made using commercial pre-packed GSH-Sepharose resins. Generating this column directly on an HPLC allowed for fast EDC addition and wash followed by incubation with GST-FcγRIIIa, which is an advantage due to the highly labile nature of EDC.¹¹ In addition, the modification of GSH with EDC and crosslinking of GST-FcγRIIIa could be monitored directly by absorbance in real time at A214 or A280.

### Enrichment of Nonfucosylated IgG1

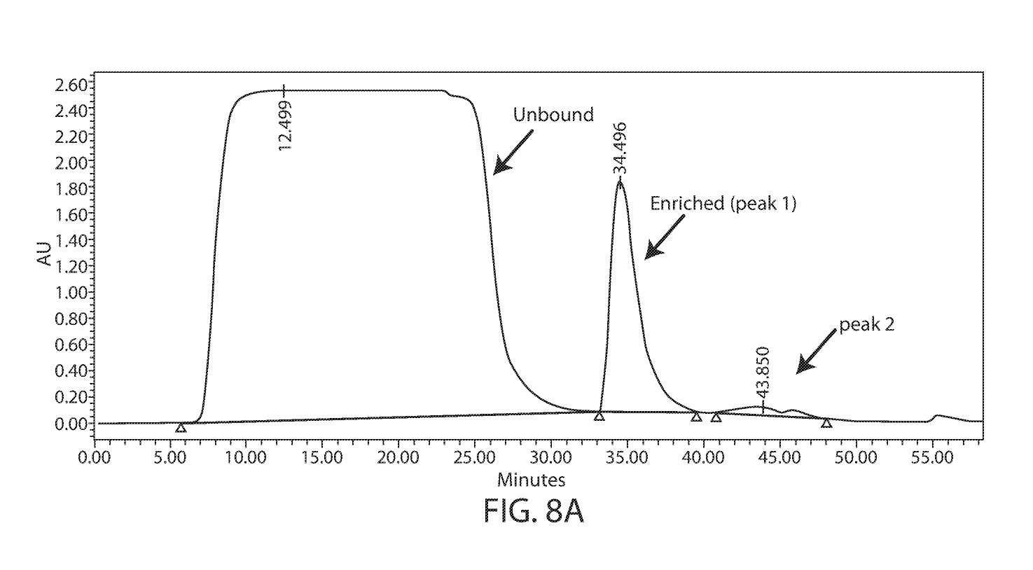

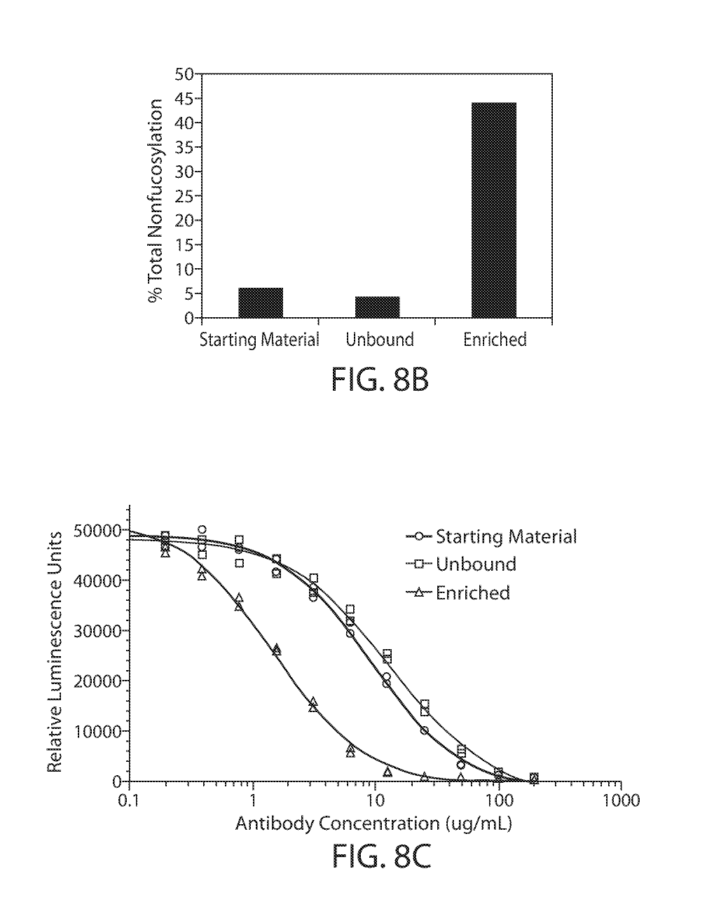

To demonstrate that the GST-FcγRIIIa affinity columns were functional, specific and could be used for glycoform variant isolations, these columns were used to isolate and enrich for stronger and weaker binding IgG1 variants, such as nonfucosylated and fucosylated IgG1 respectively. To enrich for nonfucosylated IgG1, the column was loaded with a 10 mL solution of 2.5 mg/mL IgG1 in 100 mM phosphate, 50 mM NaCl pH 7.0. To remove unbound and weakly bound IgG1, the column was washed with the same equilibration buffer. Finally, the bound IgG1 was eluted from the column with 50 mM citrate, 100 mM NaCl pH 4.2, followed by 100 mM glycine pH 3.0 at 0.5 mL/minute. This two-step low pH elution resulted in two peaks as detected by A280 (FIG. 8A). The UV elution peaks for the unbound, enriched peak 1 and smaller peak 2 were collected and analyzed for N-glycan content and by an orthogonal FcγRIIIa binding assay. The first major eluate peak contained 44% total nonfucosylated IgG1, which was 7-fold enriched in nonfucosylated N-glycan species compared to the starting material, which contained approximately 6% nonfucosylated IgG1 (FIG. 8B). The second minor eluate peak contained approximately 5-10% higher levels of nonfucosylated IgG1 than the first peak. However, there were very small amounts of protein in the second eluate peak. Furthermore, it was determined that the use of the second (lower pH) glycine buffer shortened the lifetime of the column. Therefore, this second lower pH elution step was not used in further experiments. These data corroborated the findings which previously showed that FcγRIIIa columns could be used to enrich for nonfucosylated species.³

These isolated forms of IgG1s were tested in a solution phase FcγRIIIa competition binding assay to show if there were any differential binding activities and to show the elution procedure did not disrupt their functions.⁸ As expected, the enriched nonfucosylated IgG1 eluate exhibited an increased FcγRIIIa binding activity of 620% compared to the starting material, as observed by the left shift of the competitive binding graph (FIG. 8C). In contrast, the column effluent, which contained 4.6% nonfucosylated IgG1 species compared to approximately 6% nonfucosylated IgG1 species in the starting material, exhibited a reduced FcγRIIIa binding activity of 69% compared to the starting material in the solution phase competition assay (FIG. 8C). Taken together, this study demonstrated that the immobilized GST-FcγRIIIa receptors generated using enzyme-substrate specific crosslinking were functional and specific for resolution of high (fucosylated) and very high (non-fucosylated) affinity interactions.

These experiments illustrate non-limiting examples of a simple and straightforward procedure to create an affinity resin (GST-FcγRIIIa) that is able to isolate IgGs differing in levels or types of glycosylation. Conditions were mild enough for the IgGs to retain their native functional abilities to bind to FcγRIIIa. This procedure takes advantage of the enzyme-substrate affinity of GST for GSH to bring amine groups within the GST pocket into close proximity to the carboxylic acid groups of GSH. By localizing the crosslinking to the substrate pocket of GST and removing excess crosslinker, this procedure minimizes modification of potentially critical amino acids that are required for the full function of GST fusion proteins. This methodology yielded immobilized GST-FcγRIIIa receptors that do not leach from the column and could be used to isolate enriched IgG species based on both stronger binding affinities to FcγRIIIa. This procedure can be used to support structure-function studies of different glycoforms of the IgG1s. It can also be used in commercial processes whereby control of specific forms of IgGs could either enhance potency, avidity or be more selective to their respective in vivo targets. This technique can be used when modifications of critical amino acids leads to changes in activity.

### Experimental Procedures

### Materials

Highly purified recombinant human monoclonal antibody IgG1s (Mab1 and Mab2) and the soluble extra-cellular domain of FcγRIIIa fused to glutathione S-transferase (GST-FcγRIIIa) were generated at Biogen. Deglycosylated IgG1s were generated by incubation with Peptide-N-glycosidase F (PNGase F) from Prozyme (CAT #: GKE-5006A) according to the manufacturer's instructions. Deglycosylation was confirmed by mass spectrometry. GE Healthcare NHS prepacked 1 mL columns (CAT #17-0716-01), Glutathione Sepharose 4 fast flow resin (CAT #: 17-5132-02) and pre-packed GSTrap FF 1 mL columns (CAT #: 17-5130-01) were purchased from Fisher Scientific. The filters used for the bulk resin preparation were the Ultrafree-MC-HV 0.45 μm centrifugal filters (CAT #: UFC30HV00, Merck Millipore). Concentration and buffer exchanges of protein solutions were done with Amicon Ultra 4 Centrifugal filters, 50,000 NMWL (CAT #: UFC805024, Merck Millipore) using 5 volumes at ambient temperature (18-22° C.). Buffers were prepared with sodium phosphate dibasic heptahydrate (CAT #: BP331, Fisher), sodium phosphate monobasic (CAT #: S9638, Sigma), Tris Base (CAT #: T6066, Sigma), Tris HCl (CAT #: 4103, JT Baker), NaCl (CAT #: 3627, JT Baker), citric acid monohydrate (CAT #: 0115, J T Baker), sodium acetate trihydrate (CAT #: 6131-4, Fisher), glacial acetic acid (CAT #: 9526-01, JT Baker), and glycine (CAT #: 0581, JT Baker). 1-ethyl-3-(3-dimethlyaminopropy) carbodiimide (EDC) was purchased from ForteBio (CAT#: 18-5094). SDS-PAGE polyacrylamide gels (CAT#: NP0322BOX), SDS sample buffer (CAT#: LC 2676), See Blue Pre-Stained Standard (CAT #: LC5625), MOPS running buffer (CAT#: NP0001), Simply Blue Safe Stain (CAT#: LC 6065) were purchased from Life Technologies.

### Gel Electrophoresis

Antibody and GST-FcγRIIIa were analyzed using one dimensional non-reducing SDS-PAGE electrophoresis. Samples were boiled for 5 minutes in non-reducing SDS sample buffer and approximately 2 μg of protein was loaded per lane into 4-12% Bis Tris gels. The gels were run in MOPS running buffer at 200 V constant voltage and stained with Coomassie Blue for at least 3 hours and destained with distilled water until the background was minimal.

### GST-FcγRIIIa Bulk Resin Preparation

All steps were carried out at ambient temperature. Glutathione Sepharose 4 fast flow resin was equilibrated with 5 column volumes of 100 mM phosphate 50 mM NaCl, pH 7.0. Phosphate buffered saline was removed by 10-15 second centrifugation in 0.45 μm centrifugal filter tubes using a Thermo IEC Micromax centrifuge with a fixed angle rotor at 350×g (2000 rpm) for 10-15 sec. A freshly prepared solution of 10 μM EDC in 100 mM phosphate, 50 mM NaCl pH 7.0 was immediately added to the resin and was allowed to incubate for 30 minutes. Following the incubation, the residual EDC solution was removed by a very quick buffer exchange. The resin is placed in Ultrafree-MC-HV 0.45 μm centrifugal filters and washed with 3 diavolumes of 100 mM phosphate, 50 mM NaCl pH 7.0. Then the EDC-activated resin was incubated with a solution of 3 mg/mL GST-FcγRIIIa in 100 mM phosphate 50 mM NaCl (1 mL of resin to 1 mL of 3 mg/mL GST-FcγRIIIa). Completing this step in one minute or less can be useful to minimize the inactivation of EDC. The resin was then gently rotated in a rotating shaker (Labquake/Barnstead/Thermolyne) for 1 hour. Following incubation, the resin was transferred to a 0.45 μm filter tube and centrifuged at 350×g for 10 to 15 seconds to remove the unbound GST-FcγRIIIa solution. The resin was washed with 5 diavolumes of 100 mM phosphate 50 mM NaCl, pH 7.0, 3 diavolumes of 50 mM citrate 100 mM NaCl, pH 4.2 and 3 diavolumes with 100 M glycine pH 3.0 to remove weakly bound GST-FcγRIIIa, and finally 5 diavolumes with PBS. All washes were collected and measured for protein concentration at A280 (the extinction coefficient used for GST-FcγRIIIa is 1.81). The total GST-FcγRIIIa loaded onto the resin was determined by subtracting the protein in the washes from the material originally added to the resin. Approximately 1.5 to 3.0 mg/mL of GST-FcγRIIIa was conjugated to the resin. The resin was stored in 100 mM phosphate 50 mM NaCl, 0.02% sodium azide pH 7.0 at 2-8° C. (to prevent bacterial growth).

### Prepacked GST Column Preparation

Prepacked GSTrap 1 mL columns were equilibrated with 100 mM phosphate 50 mM NaCl pH 7.0 on a Waters Alliance 2697 Separation Module with a model 2784 dual wave detector. Following equilibration, 200 μl of 400 mM 1-ethyl-3-(dimethylaminopropyl) carbodiimide (EDC) dissolved in H2O was injected onto the column. The flow of EDC was monitored in the flow through with A214 or A280. Immediately following the absorbance return to baseline, 9 mL of 1 mg/mL GST-FcγRIIIa in 50 mM phosphate pH 7.0 buffer was loaded onto the column.

After loading the GST-FcγRIIIa, the column was washed with 100 mM phosphate, 50 mM NaCl, pH 7.0 until the UV trace returned to base line. Next, to remove any unbound GST-FcγRIIIa, the column was washed with 100 mM citrate, 100 mM NaCl, pH 4.0 for 40 minutes at 0.5 mL/minute, followed by 100 mM glycine, pH 3.0 for 40 minutes, followed by 100 mM sodium acetate, 500 mM NaCl, pH 4.0, and finally with 100 mM phosphate 50 mM NaCl pH 7.0 for 30 minutes. The column was stored in 100 mM phosphate 50 mM NaCl, 0.02% Sodium Azide pH 7.0 (to prevent bacterial growth). The amount of GST-FcγRIIIa loaded was between 1.5 and 3.0 mg of GST-FcγRIIIa per mL resin.

### Prepacked NHS Column Preparation

Prepacked 1 mL NHS columns were equilibrated with 100 mM phosphate 50 mM NaCl pH 7.0 at 0.5 mL/minute. Following equilibration, 2.2 mg of GST-FcγRIIIa was loaded and recycled over the column for 30 minutes. Next, the column was washed for 5 minutes at 0.5 mL/minute with 100 mM phosphate 50 mM NaCl pH 7.0. Unreacted NHS groups were quenched with 2 M Tris pH 7.0 at 0.5 mL/minute for 30 min and then allowed to incubate at 4° C. overnight to saturate any remaining amine binding sites. Next, to remove any unbound GST-FcγRIIIa, the column was washed with 100 mM citrate, 100 mM NaCl, pH 4.0 for 40 minutes, followed by 100 mM glycine, pH 3.0 for 40 minutes, then with 100 mM sodium acetate, 500 mM NaCl, pH 4.0, and finally with 100 mM phosphate 50 mM NaCl pH 7.0 for 30 minutes. The column was stored in 100 mM phosphate 50 mM NaCl, 0.02% Sodium Azide pH 7.0 (to prevent bacterial growth). All unbound and buffer washes were collected and the total binding of the GST-FcγRIIIa was determined by subtracting the unbound and washes from the starting material added to the column. Approximately 0.93 mg GST-FcγRIIIa was covalently bound to the 1 mL column.

### Fluorescence and Phase Contrast Imaging of GST-FcγRIIIa Resin

GSH-Sepharose was cross-linked to GST-FcγRIIIa with EDC or without EDC at room temperature for 30 minutes. Following the crosslinking and quenching reactions as described above, the resins were washed with 50 mM citrate buffer 100 mM NaCl, pH 4.2 buffer, pH 3.0 phosphate buffer pH 9.2, 10 mM glutathione and 2% SDS. After these washings, the resins were washed in 100 mM phosphate 50 mM NaCl pH 7.0 and incubated with FITC-conjugated anti-CD16 antibody (CAT.#: MHCD1601, Life Technologies) and allowed to rotate for 60 minutes at 4° C. Following the incubation, the resins were washed three times with 100 mM phosphate 50 mM NaCl pH 7.0 to remove excess anybody. Fluorescence and phase contrast images were captured through a 10× objective lens using an inverted reflected light microscope (Model#: CKX41, Olympus), with a fluorescent light source (Model#: XCITE Series 120, EXFO Photonic Solutions) and with a fluorescence camera (Model#: DP71, Olympus). A 1.5 sec exposure time was used for fluorescence imaging of (−) EDC and (+) EDC treated sepharose.

### Enrichment of Monoclonal Antibody IgG1

The column was first equilibrated with 100 mM phosphate, 50 mM NaCl, pH 7.0 at 0.5 mL/minute. Next, 10 mL of a 2.5 mg/mL solution IgG was loaded on to the column and washed with the same equilibration buffer. Following the return of the UV to baseline, bound IgG was eluted from the column with 50 mM citrate, 100 mM NaCl pH 4.2 (enriched peak 1), followed by 100 mM glycine pH 3.0 (peak 2) at 0.5 mL/minute. The UV elution peaks were collected in polypropylene tubes containing 2 mL of 2 M Tris pH 7.0 (to immediately neutralize the solution). After the run, the column was re-equilibrated with 100 mM phosphate 50 mM NaCl, pH 7.0. The citrate and glycine eluted fractions were buffer exchanged by centrifugation with 50,000 NMWL filter tube and resuspension in 100 mM phosphate 50 mM NaCl.

### N-Glycan Analysis

The N-glycan profile of the monoclonal antibodies was analyzed using the Prozyme GlykoPrep Digestion Module (GS96-RX) and the Prozyme GlykoPrep Cleanup Module (GS96-CU). Briefly, 50 μg of the monoclonal antibodies were used in each preparation. The N-glycans were removed by digestion with N-Glycanase for one hour at 50° C. and then separated from the monoclonal antibody with the RX tips supplied in the Digestion Module and then labeled with two 2 amino benzamide (2-AB). Excess 2-AB was removed by passing the reactions solution through the Clean Up tips supplied in the Cleanup Module. The labeled N-glycan samples were analyzed on the HILIC column (BEH Glycan Column, 2.1 mm×150 mm, 186004742) on a Waters UPLC with a fluorescence detector. Samples were run on a 24 minute gradient of 25% 0.1 M ammonium formate, 75% acetonitrile pH 4.5 to 100% 0.1 M ammonium formate pH 4.5 at 60° C.

% Total Nonfucosylation=G0%+*G1/G1F−GlcNAc %+G0−GlcNac %+G1−GlcNAc %+G2%+Man 3+Man5%+Man6+Man7%+Man8%+Man9%+A1%+A2%+*A1−GlcNAc %

### AlphaScreen Competitive Binding Assay

To determine the relative affinity for FcγRIIIa binding, a competitive AlphaScreen assay was used as previously described.⁸ In brief, samples were diluted in assay buffer (1×PBS/0.01% Tween 20/0.1% BSA) and added to the assay plate at a starting concentration of 200 μg/mL. Next, GST-FcγRIIIa was added to the plate at a final concentration of 0.17 μg/mL and GSH-coated donor beads and antibody conjugated acceptor beads (donor and acceptor beads from Perkin Elmer; IgG1 conjugated acceptor beads were made for Biogen by Perkin Elmer) were added to the plate at a final concentration of 3.3 μg/mL. After shaking the plate for 2 hours at 22° C.±1° C., luminescence was read using an EnVision plate reader. The data was analyzed using SoftMax Pro, and IC₅₀ values were used to determine relative binding activity.

### Example 2: A Simple Enzyme-Substrate-Localized Conjugation Method to Generate PEGylated Functional GST-Fusion Proteins

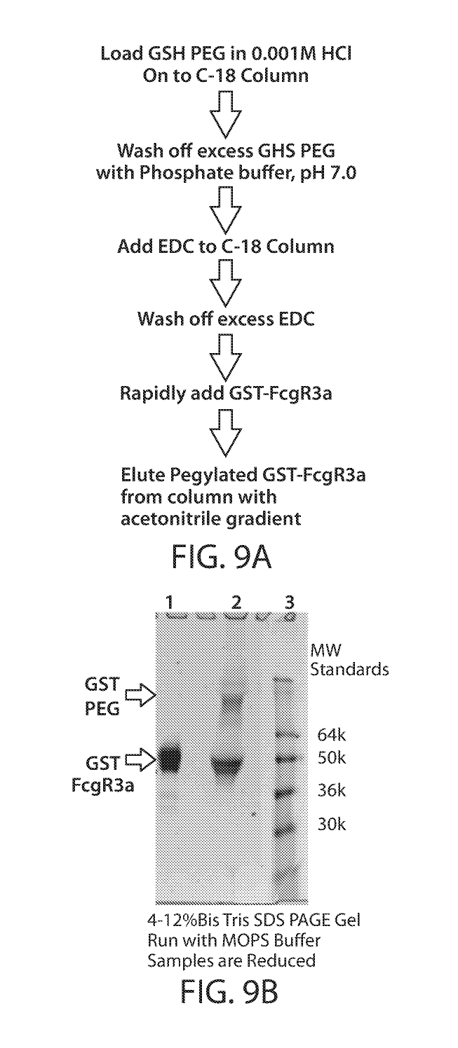

FIGS. 9A and 9B illustrate an example of attaching a protein to one or more polymers, for example, to improve a biophysical or pharmacokinetic property of the therapeutic protein.

FIG. 9A illustrates a PEGylation procedure of GST-FcγRIIIa to GSH-PEG. Equilibrate a 50 mm×4.6 mm C-18 column with 0.001 M HCl. Inject 300 μl of 22 mg/mL GSH-PEG in 0.001 M HCl, Wash away excess GSH-PEG with 100 mM phosphate 50 mM NaCl pH 7.0, Add 400 μl of EDC (400 mM). Briefly wash with 100 mM phosphate, 50 mM NaCl pH 7.0 to remove excess EDC. Quickly load 200 μl of 3.9 mg/mL GST-FcγR3a in 100 mM phosphate, 50 mM NaCl pH 7.0. Wash with 100 mM phosphate, 50 mM NaCl pH 7.0. Elute GST-FcγRIIIa coupled to GSH-PEG with a 10 minutes 0-100% acetonitrile gradient.

FIG. 9B illustrates a non-reducing 4-12% Bis Tris SDS-PAGE stained with coomassie blue (1) GST-FcγRIIIa, 2 μg, (2) GST-FcγRIIIa coupled to GSH-PEG, 2 μg, (3) Molecular weight markers (as shown).

### REFERENCES

(1) Punna S., Kaltgrad E., Finn M. G., (2005) “Clickable” agarose for affinity chromatography, Bioconjugate Chemistry 1536-1541.
(2) Scholthauer T., Rueger P., Stracke J. O., Hertenberger H., Fingas F., Kling L., Emrich T., Drabner G., Seeber S., Auer J., Koch S., Papadimitrou A., (2013) Analytical FcRn affinity chromatography for functional characterization of monoclonal antibodies. MAbs Journal 576-586.
(3) Bolton G., Ackerman M. E., Boesch A. W., (2013) Separation of nonfucosylated antibodies with immobilized FcγRIII receptors. Biotechnol. Prog. 825-828.
(4) E. W. Lin, N. Boehnke, H. D. Maynard, (2014) Protein-Polymer Conjugation via Ligand Affinity and Photoactivation of Glutathione S-Transferase, Bioconjugate Chemistry 1902-1909.
(5) Terpe K., (2003) Overview of tag fusion proteins: from molecular and biochemical fundamentals to commercial systems. Appl Microbiol Biotechnol 60: 523-533.
(6) Ferrara C., Grau S., Jager C., Sondermann P., Brunker P., Waldhauer I., Hennig M., Ruf A., Rufer A. C., Stihle M., Umana P., Benz J., (2011) Unique carbohydrate-carbohydrate interactions are required for high affinity binding between FcγRIII and antibodies lacking core fucose. Proc Natl Acad Sci USA 12669-74.
(7) Zhou, Y., Guo, T., Tang G., Wu H., Wong N. K., Pan Z., (2014) Site-Selective Protein Immobilization by Covalent Modification of GST Fusion Proteins, Bioconjugate Chemistry 1911-1915.
(8) Houde D., Peng Y., Berkowitz S. A., Engen J. R., (2010) Post-translational modifications differentially affect IgG1 conformation and receptor binding, Molecular & Cellular Proteomics 9.8.
(9) Deu E., Verdoes M., Bogyo M., (2012) New approaches for dissecting protease functions to improve probe development and drug discovery. Nature Structural & Molecular Biology 19: 1.
(10) Prade, L., Huber, R., Manoharan, T. H., Fahl, W. E., Reuter, W., (1997) Structures of class pi glutathione S-transferase from human placenta in complex with substrate, transition-state analogue and inhibitor. Structure 5: 1287-1295. PDB 1AQW.
(11) Hermanson, G. T. (2013) Bioconjugate Techniques. pp 259-266, Chapter 4, Academic Press, New York.
(12) Stierand, K., Maab, P., Rarey, M. (2006) Molecular complexes as a glance: Automated generation of two-dimensional complex diagrams. Bioinformatics 22: 1710-1716.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method of connecting a protein of interest to a substrate, the method comprising:
(i) contacting a crosslinking agent to a first binding partner under first reaction conditions suitable for forming a first covalent bond between the crosslinking agent and the first binding partner, wherein the first binding partner is attached to a substrate, and
(ii) contacting a second binding partner to the first binding partner under second reaction conditions suitable for forming a second covalent bond between the crosslinking agent and the second binding partner,
wherein the first and second binding partners bind to each other under the second reaction conditions, and wherein the second binding partner is attached to a protein of interest.

2. The method of claim 1, wherein the substrate is a solid support.

3. The method of claim 2, wherein the solid support is a resin.

4. The method of claim 2, wherein the solid support comprises sepharose, agarose, silica, or polystyrene-divinyl-benzene.

5. The method of claim 4, wherein the solid support is a sepharose bead.

6. The method of claim 1, wherein the substrate is a polymer.

7. The method of claim 4, wherein the polymer is polyethylene glycol (PEG).

8. The method of any prior claim, wherein the crosslinking agent is a zero-length cross-linker.

9. The method any of claims 1-8, wherein the crosslinking agent covalently links a carboxylic acid to a primary amine.

10. The method claim 9, wherein the crosslinking agent is 1-ethyl-3-(dimethylaminopropyl) carbodiimide (EDC) or dicyclohexylcarbodiimide (DCC).

11. The method any of claims 1-8, wherein the crosslinking agent covalently links a primary amine to a primary amine.

12. The method of claim 11, wherein the crosslinking agent is a N-hydroxysuccinimide (NHS)-ester crosslinker, or disuccinimidyl suberate (DSS).

13. The method of any prior claim, wherein unbound crosslinking agent is removed after contacting the first binding partner in i) and before contacting the second binding partner in ii).

14. The method of any prior claim, wherein the first binding partner is covalently attached to the substrate.

15. The method of any prior claim, wherein the first binding partner is non-covalently attached to the substrate.

16. The method of any prior claim, wherein the first binding partner is glutathione (GSH) and the second binding partner is glutathione S-transferase (GST), structural maintenance of chromosomes 1 (SMC1), or RalA Binding Protein 1 (RALBP1).

17. The method of any prior claim, wherein the second binding partner is a polypeptide.

18. The method of claim 17, wherein the protein of interest and the second binding partner are in the form of a fusion protein.

19. The method of any prior claim, wherein the protein of interest is a therapeutic protein.

20. The method of claim 19, wherein the therapeutic protein is a therapeutic antibody, enzyme, hormone, or growth factor.

21. The method of any prior claim, wherein the protein of interest is an affinity tag capable of binding to a molecule of interest.

22. A method of immobilizing an affinity protein to a solid support, the method comprising:
(i) contacting a first binding partner with a crosslinking agent, wherein the first binding partner is attached to a solid support, and
(ii) contacting the first binding partner of (i) with a second binding partner, wherein the second binding partner is attached to the affinity protein.

23. The method of claim 22, wherein the first binding partner is glutathione (GSH) and the second binding partner is glutathione S-transferase (GST).

24. The method of claim 22, wherein the first binding partner is streptavidin, and the second binding partner is biotin.

25. The method of claim 22, wherein the first binding partner is glutathione (GSH) and the second binding partner is, structural maintenance of chromosomes 1 (SMC1), or RalA Binding Protein 1 (RALBP1).

26. The method of any one of claims 22-25, wherein the crosslinking agent is a zero-length crosslinker.

27. The method of any one of claims 22-26, wherein the zero-length crosslinker links a carboxylic acid to a primary amine.

28. The method of any one of claims 22-27, wherein the zero-length crosslinker is 1-ethyl-3-(dimethylaminopropyl) carbodiimide (EDC).

29. The method of any one of claims 22-27, wherein the zero-length crosslinker is dicyclohexylcarbodiimide (DCC).

30. The method of any one of claims 22-25, wherein the crosslinking agent links a primary amine to a primary amine.

31. The method of claim 30, wherein the crosslinking agent is a N-hydroxysuccinimide (NHS)-ester crosslinker.

32. The method of claim 31, wherein the crosslinker is disuccinimidyl suberate (DSS).

33. The method of any one of claims 22-32, wherein the solid support comprises a synthetic resin.

34. The method of any one of claims 22-32, wherein the solid support comprises sepharose, agarose, silica, or polystyrene-divinyl-benzene.

35. The method of any one of claims 22-32, wherein the solid support comprises sepharose beads.

36. The method of any one of claims 22-35, wherein the solid support is arranged in a column.

37. The method of any one of claims 22-36, wherein the affinity protein comprises a receptor.

38. The method of any one of claims 22-37, wherein the affinity protein comprises an Fc gamma receptor IIIa (FcgRIIIa), Fc gamma receptor IIa, or a fragment thereof.

39. The method of any one of claims 22-38, further comprising a wash step between (i) and (ii).

40. An affinity resin comprising:
(a) a solid support material bound to glutathione (GSH), and
(b) an affinity protein bound to glutathione S-transferase (GST), wherein the GSH and GST are covalently linked by an amide bond.

41. The affinity resin of claim 40, wherein Lysine 44 and/or Glutamine 51 of GST is covalently linked to GSH by an amide bond.

42. An affinity chromatographic device comprising:
(a) a solid support material bound to glutathione (GSH), and
(b) an affinity protein bound to glutathione S-transferase (GST), wherein the GSH and GST are covalently linked by an amide bond.

43. The affinity chromatographic device of claim 42, wherein the affinity chromatographic device comprises:
a chromatographic column containing,
the solid support material bound to glutathione (GSH) of (a), and
the affinity protein bound to glutathione S-transferase (GST) of (b).

44. A method of purifying a protein, the method comprising:
(a) contacting a sample comprising the protein with the affinity resin of claim 40 or 41, or the affinity chromatographic device of claim 42 or 43,
(b) contacting the resin or the affinity chromatography separation device of (a) with a wash buffer, and
(c) eluting the protein from the affinity resin or affinity chromatography separation device.
